(19) United States
(12) Patent Application Publication
(10) Pub. No.: US 2021/0380602 A1
(43) Pub. Date: Dec. 9, 2021

(54) NOVEL PYRROLE AND PYRIDONE DERIVATIVES AND USES THEREOF

(71) Applicant: VIRGINIA COMMONWEALTH UNIVERSITY, Richmond, VA (US)

(72) Inventors: Jo-Ann JEE, Richmond, VA (US); Narendar Reddy GADE, Richmond, VA (US); Thomas D. ROPER, Richmond, VA (US)

(21) Appl. No.: 17/286,177

(22) PCT Filed: Jul. 31, 2019
(86) PCT No.: PCT/US2019/044382

§ 371 Date: Apr. 16, 2021

Related U.S. Application Data
(60) Provisional application No. 62/747,739, filed on Oct. 19, 2018.

(51) Int. Cl. C07D 498/14

(52) U.S. Cl. CPC C07D 498/14

(57) ABSTRACT

Pyrrole and pyridine derivatives and methods of synthesizing the derivatives/analogs are provided. In particular, the compounds are useful for the treatment of antiviral infections such as HIV and influenza.

### FIELD OF THE INVENTION

The invention is generally related to methods to synthesize pyrrole and pyridine derivatives/analogs. In particular, the derivatives/analogs are useful for the treatment of antiviral infections such as HIV and influenza.

### BACKGROUND OF THE INVENTION

Antiretroviral (ARV) treatments often involve combination therapy which plays a role in fighting drug resistance. Integrase inhibitors (INIs) are used in combination therapy and their mechanism of action is to block the viral integrase enzyme from inserting the viral genome into the host DNA. Preventing this crucial step in retroviral replication will inhibit the spread of the virus. Dolutegravir is a second-generation HIV integrase inhibitor that was approved by the Food and Drug Administration (FDA) in 2013. Dolutegravir is currently applied in HIV combination therapy and it has been reported to have a better mutation resistance profile compared to first-generation HIV integrase inhibitors raltegravir and elvitegravir. Other INIs such as cabotegravir is in phase III clinical trials, and bictegravir has been recently approved (2018) by the FDA for combination drug therapy. With the increase in drug resistance progression, there is a need for the development of new HIV integrase inhibitors.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

In an aspect of the invention, a compound is provided having the formula II:

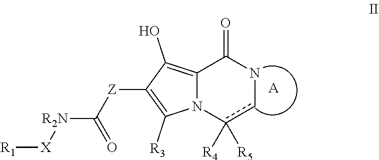

where ring A is an optionally substituted heterocycle
where Z is selected from the group consisting of:

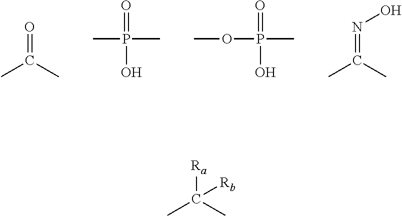

where Rₐ and R_{b} are independently substituted hydrogen, OH, C₁-C₁₀ alkyl or C₁-C₁₀ alkoxide
where X is selected from the group consisting of a single bond, a heteroatom or a heteroatomic group selected from the group consisting of O, S, SO, SO₂ and NH, a C₁-C₆ alkylene, a C₁-C₆ heteroalkylene, a C₂-C₆ alkenylene or C₂-C₆ heteroalkenylene;
where R₁ is an optionally substituted aryl;
where R₂ is a hydrogen or C₁-C₁₀ alkyl;
where R₃ is selected from the group consisting of a hydrogen, a halogen, a hydroxy, an optionally substituted C₁-C₁₀ alkyl, an optionally substituted C₃-C₈ cycloalkyl, an optionally substituted C₂-C₈ alkenyl, an optionally substituted C₁-C₁₀ alkoxy, an optionally substituted C₂-C₈ alkenyloxy, an optionally substituted aryl, optionally substituted aryloxy, an optionally substituted heterocyclic group, an optionally substituted heterocycleoxy, an optionally substituted amino, an optionally substituted carbamoyl, and an optionally substituted carbamoylcarbonyl;
where R₄ and R₆ are independently a hydrogen, a hydroxyl, an optionally substituted C₁-C₁₀ alkyl, an optionally substituted C₁-C₁₀ heteroalkyl, an optionally substituted C₂-C₈ alkenyl, an optionally substituted C₁-C₁₀ alkoxy, an optionally substituted C₂-C₈ alkenyloxy, an optionally substituted aryl, an optionally substituted aryloxy, an optionally substituted heterocycle, an optionally substituted heterocycleoxy, an optionally substituted amino, an optionally substituted carbamoyl, an optionally substituted carbamoylcarbonyl, an optionally substituted phosphoric acid moiety, optionally substituted C₃-C₈ cycloalkyl, an optionally substituted heteroalkyl, an optionally substituted aryl, and an optionally substituted heteroaryl, a substituted aryl, aralkyl, C₁-C₁₀ alkyl, C₁-C₁₀ heteroalkyl, hydroxyl, or amino, wherein the substituted aryl, aralkyl, C₁-C₁₀ alkyl, C₁-C₁₀ heteroalkyl, hydroxyl, amino is substituted with an optionally substituted phosphoric acid moiety, a substituted or unsubstituted C₁-C₁₀ heteroalkyl and the substituted or unsubstituted C₁-C₁₀ heteroalkyl comprises one or more of O, S, SO, SO₂, or NR₅, and where [symbol] represents a single or double bond, wherein if [symbol] is a double bond, then R₆ is not present.

In another aspect, these new compounds possess anti-viral properties useful for the treatments of viral diseases including influenza and HIV.

In further aspect of the invention, the compound of formula II-a is

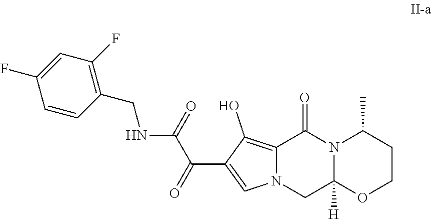 [SMILES: C[C@@H]1CCO[C@H]2Cn3cc(C(=O)C(=O)NCc4ccc(F)cc4F)c(O)c3C(=O)N12]

or pharmaceutically acceptable salt of solvate thereof.

Another aspect of this invention to provide a method for the preparation of compound of formula II, and dolutegravir from a common precursor.

In another aspect, the common precursor is compound of formula 18

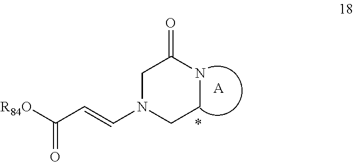

where A is an optionally substituted heterocycle
R₈₄ is a hydrogen or C₁-C₁₀ alkyl.

In another aspect, an important intermediate compound for creating compounds of the claimed invention is a compound of formula 17

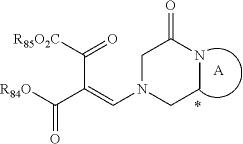

where A is an optionally substituted heterocycle,
and R₈₄ and R₈₅ are independently hydrogen or C₁-C₁₀ alkyl e.g. methyl, ethyl, propyl, isopropyl, etc.

In another aspect, acompound of the claimed invention is a compound of formula 20:

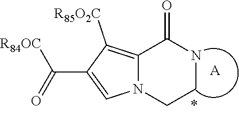

where A is an optionally substituted heterocycle,
and R₈₄ and R₈₅ are independently hydrogen or C₁-C₁₀ alkyl e.g. methyl, ethyl, propyl, isopropyl, etc.

### DETAILED DESCRIPTION

The following description and examples illustrate some exemplary embodiments of the disclosed invention in detail. Those of skill in the art will recognize that there are numerous variations and modifications of this invention that are encompassed by its scope. Accordingly, the description of exemplary embodiments should not be deemed to limit the scope of the present invention.

### Definition:

CaO: calcium oxide
Ca(OMe)₂: calcium methoxide
DBU: 1,8-Diazabicyclo[5.4.0]undec-7-ene
DCC: N,N′-dicyclohexylcarbodiimide
DCM: dichloromethane
DIPEA: N,N-diisopropylethylamine.
DMAP: (4-dimethylaminopyridine)
DMF: dimethylformamide
DMSO: dimethyl sulfoxide
EDC (also EDAC or EDCI): 1-Ethyl-3-(3-dimethylaminopropyl)carbodiimide
Et₃N: triethyl amine
EtOH: ethanol
HATU: (1-[Bis(dimethylamino)methylene]-1H-1,2,3-triazolo[5-b]pyridinium 3-oxid hexafluorophosphate)
KOH: potassium hydroxide
K₃PO₄: potassium phosphate
LDA: lithium diisopropylamide
LiHMDS: lithium bis(trimethylsilyl)amide is a lithiated organosilicon compound with the formula LiN(SiMe₃)₂
LiOMe: lithium methoxide
MeCN: acetonitrile
MeOH: methanol
MgBr₂: magnesium bromide
MgBr.OEt₂: magnesium bromide ethyl etherate
MgCl₂: magnesium chloride
Mg(OMe)₂: magnesium methoxide
Mg(OtBu)₂: magnesium t-butoxide
NaH: sodium hydride
NaOH: sodium hydroxide
NaOMe: sodium methoxide
NMP: N-methyl-2-pyrrolidine
THF: tetrahydrofuran

As used herein, any “R” group(s) such as, without limitation, R, R_{I}, R₂, R₃, R₄, R₅, R₆, R₇, R₈, R₉, R₁₀, R₁₁, R₁₂, R₁₃, R₁₄, R₁₅, and so on represent substituents that can be attached to the indicated atom. An R group may be substituted or unsubstituted. If two “R” groups are described as being “taken together” the R groups and the atoms they are attached to can form a cycloalkyl, aryl, heteroaryl, or heterocycle.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfmyl, sulfonyl, haloalkyl, haloalkoxy, trihalomethanesulfonyl, trihalomethanesulfonamido, an amino, a mono-substituted amino and a di-substituted amino group, and protected derivatives thereof.

As used herein, “Cₐ to C_{b},” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—, if no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

As used herein, “alkyl” refers to a straight or branched hydrocarbon chain that includes a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. An alkenyl group may be unsubstituted or substituted.

As used herein, “alkynyl” refers to an alkyl group that contains in the straight or branched hydrocarbon chain one or more triple bonds. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain, for example, 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

As used herein, “cycloalkynyl” refers to a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms, that is, an element other than carbon, including but not limited to, nitrogen, oxygen and sulfur. The number of atoms in the ring(s) of a heteroaryl group can vary. For example, the heteroaryl group can contain, for example, 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine. A heteroaryl group may be substituted or unsubstituted.

As used herein, “heterocyclyl” or “heteroalicyclyl” refers to e.g. a three-, four-, five-, six-, seven-, eight-, nine-, ten-, etc. up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1 ,3-dioxane, 1,4-dioxane, 1 ,2-dioxolane, 1,3-dioxolane, 1 ,4-dioxolane, 1,3-oxathiane, 1,4-oxathiin, 1,3-oxathiolane, 1,3-dithiole, 1,3-dithiolane, 1 ,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholme, thiamorpholme sulfoxide, thiamorpholme sulfone, and their benzo-fused analogs (e.g., benzimidazolidinone, tetrahydroquinoline, 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl, and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, and imidazolylalkyl, and their benzo-fused analogs. A “(heteroalicyclyl)alkyl” and “(heterocyclyl)alkyl” refer to a heterocyclic or a heteroalicyclylic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl)methyl, (piperidin-4-yl)ethyl, (piperidin-4-yl)propyl, (tetrahydro-2H-thiopyran-4-yl)methyl, and (1 ,3-thiazinan-4-yl)methyl.

As used herein, “alkoxy” refers to the formula —OR wherein R is an alkyl, an alkenyl, an alkynyl, a cycloalkyl, a cycloalkenyl or a cycloalkynyl is defined as above. A non-limiting list of alkoxys is methoxy, ethoxy, n-propoxy, 1-methylethoxy (isopropoxy), n-butoxy, iso-butoxy, sec-butoxy and tert-butoxy. An alkoxy may be substituted or unsubstituted.

As used herein, “acyl” refers to a hydrogen, alkyl, alkenyl, alkynyl, or aryl connected, as substituents, via a carbonyl group. Examples include but are not limited to formyl, acetyl, propanoyl, benzoyl, and acryl. An acyl may be substituted or unsubstituted.

As used herein, “hydroxyalkyl” refers to an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxy ethyl. A hydroxyalkyl may be substituted or unsubstituted.

As used herein, “halogenated alkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “halogenated alkoxy” refers to an alkoxy group in which one or more of the hydrogen atoms are replaced by a halogen (e.g., mono-haloalkoxy, di-haloalkoxy and tri-haloalkoxy, etc.). Such groups include but are not limited to, chloromethoxy, fluoromethoxy, difluoromethoxy, trifluoromethoxy, 1-chloro-2-fluoromethoxy, and 2-fluoroisobutoxy. A haloalkoxy may be substituted or unsubstituted.

As used herein, “aryloxy” and “arylthio” refers to RO— and RS—, in which R is an aryl, such as but not limited to phenyl. Both an aryloxy and arylthio may be substituted or unsubstituted.

A “sulfenyl” or “thio” group refers to an “—SR” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted. The term “sulfenyl” or “thio” includes, but is not limited to an -SH group (also referred to as a “thiol” group) as well as an —SRA group (also referred to as a “thioether” when RA is not hydrogen).

A “sulfonyl” group refers to an “SO₂R” group in which R can be the same as defined with respect to sulfenyl. A sulfonyl may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein X is a halogen. A “trihalomethanesulfonamido” group refers to an “X₃CS(O)₂N(RA)-” group wherein X is a halogen and RA is hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl.

The term “amino” as used herein refers to a —N(R)₂ group, wherein R is independently selected from the group consisting of hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An amino may be substituted or unsubstituted. The term “amino” includes, but is not limited to a —NH₂ group (also referred to as an “ammonium” group), a —NHR group (also referred to as a “secondary amine” when R is not hydrogen), or a —NR, group (also referred to as a “tertiary amine” when R is not hydrogen). [0065]. As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An S-sulfonamido may be substituted or unsubstituted.

An “N-sulfonamido” group refers to a “RSO₂N(R_{A})—” group in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-sulfonamido may be substituted or unsubstituted.

The term “halogen atom” or “halogen” as used herein, means any one of the radio-stable atoms of column 7 of the Periodic Table of the Elements, such as, fluorine, chlorine, bromine and iodine.

Where the numbers of substituents is not specified (e.g. haloalkyl), there may be one or more substituents present. For example “haloalkyl” may include one or more of the same or different halogens. As another example, “C1-C3 alkoxyphenyl” may include one or more of the same or different alkoxy groups containing, for example, one, two or three atoms.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline foil is (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Certain Compounds

The present disclosure provides novel pyrrole and pyridone derivatives and methods of the preparation of the pyrrole and pyridone derivatives. The derivatives are used, for example, for the treatment of anti-viral infections such as HIV and influenza.

By “derivative” we mean a chemical compound that can be produced from another compound via one or more chemical reactions, such as replacement of heterocycles, H etc. by an alkyl, acyl, or another heterocycle, etc. Derivatives of a compound may also be referred to as modified forms of the compound. The synthesis of a derivative of a compound may proceed by modifying the compound directly, or by another synthetic route. Either way, the derivatized product typically has a core structure similar to that of the original compound, but one or more functional groups of the original compound have been replaced by different functional groups

By pyrrole derivatives, we mean a bicyclic compound of formula II, pharmaceutically acceptable salt, or solvate thereof,

where:

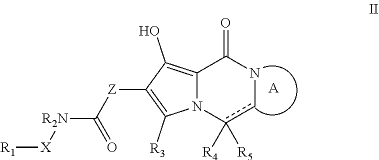

A is an optionally substituted heterocycle or heterocarbocycle;

Z is selected from the group consisting of:

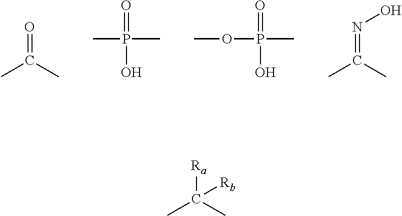

Rₐ and R_{b} are independently substituted hydrogen, OH, C₁-C₁₀ alkyl or C₁-C₁₀alkoxide

X is selected from the group consisting of a single bond, a heteroatom or a heteroatomic group selected from the group consisting of O, S, SO, SO₂ and NH, a C₁-C₆ alkylene, a C₁-C₆ heteroalkylene, a C₂-C₆ alkenylene or C₂-C₆ heteroalkenylene;

R₁ is an optionally substituted aryl;

R₂ is a hydrogen or C₁-C₁₀ alkyl;

R₃ is selected from the group consisting of a hydrogen, a halogen, a hydroxy, an optionally substituted C₁-C₁₀ alkyl, an optionally substituted C₃-C₈ cycloalkyl, an optionally substituted C₂-C₈ alkenyl, an optionally substituted C₁-C₁₀ alkoxy, an optionally substituted C₂-C₈ alkenyloxy, an optionally substituted aryl, optionally substituted aryloxy, an optionally substituted heterocyclic group, an optionally substituted heterocycleoxy, an optionally substituted amino, an optionally substituted carbamoyl, and an optionally substituted carbamoylcarbonyl;

R₄ and R₆ are independently a hydrogen, a hydroxyl, an optionally substituted C₁-C₁₀ alkyl, an optionally substituted C₁-C₁₀ heteroalkyl, an optionally substituted C₂-C₈ alkenyl, an optionally substituted C₁-C₁₀ alkoxy, an optionally substituted C₂-C₈ alkenyloxy, an optionally substituted aryl, an optionally substituted aryloxy, an optionally substituted heterocycle, an optionally substituted heterocycleoxy, an optionally substituted amino, an optionally substituted carbamoyl, an optionally substituted carbamoylcarbonyl, an optionally substituted phosphoric acid moiety, optionally substituted C₃-C₈ cycloalkyl, an optionally substituted heteroalkyl, an optionally substituted aryl, and an optionally substituted heteroaryl, a substituted aryl, aralkyl, C₁-C₁₀ alkyl, C₁-C₁₀ heteroalkyl, hydroxyl, or amino, wherein the substituted aryl, aralkyl, C₁-C₁₀ alkyl, C₁-C₁₀ heteroalkyl, hydroxyl, amino is substituted with an optionally substituted phosphoric acid moiety, a substituted or unsubstituted C₁-C₁₀ heteroalkyl and the substituted or unsubstituted C₁-C₁₀ heteroalkyl comprises one or more of O, S, SO, SO₂, or NR₅; and

[symbol] represents a single or double bond, wherein if [symbol] is a double bond, then R₆ is not present.

In one embodiment, a compound of formula II, pharmaceutically acceptable salt, or solvate thereof is of the formula:

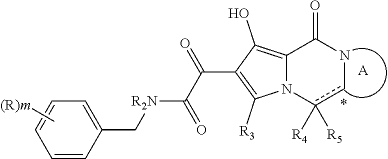

where,

* indicates an R- or S-configuration,

R is independently selected from halogen e.g. F, Cl or Br and Substituent group S1, where substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C ₁-C ₁₀ alkyl, C₁-C₁₀ alkoxy, carbamoyl optionally substituted with mono- or di-C₁-C₁₀ alkyl, optionally substituted C₁-C₁₀ alkyl sulfonyl amino, halogenated C₁-C₁₀ alkoxy and hydroxy C₁-C₁₀ alkyl; and

R₂ is independently substituted hydrogen or C₁-C₁₀ alkyl;

R₃, R₄ and R₆ are as defined above;

m is0, 1, 2,or 3;

Ring A is an optionally substituted 5-7 membered carbocycle or optionally substituted heterocycle, where ring A is one of the following:

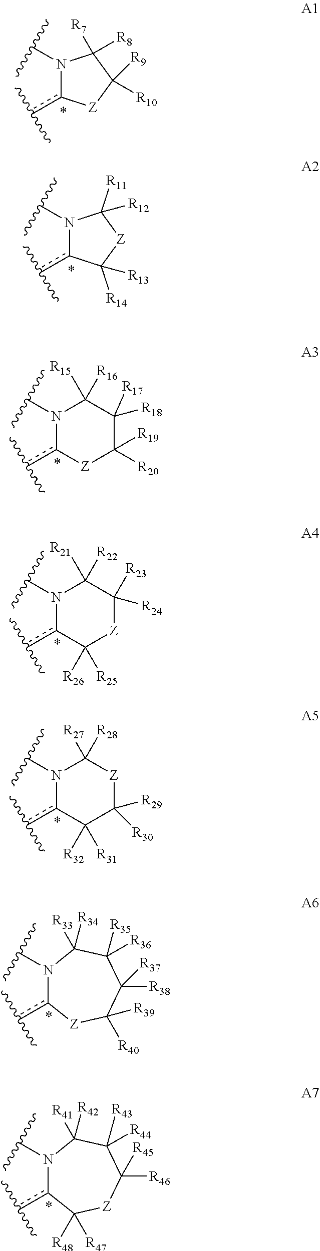

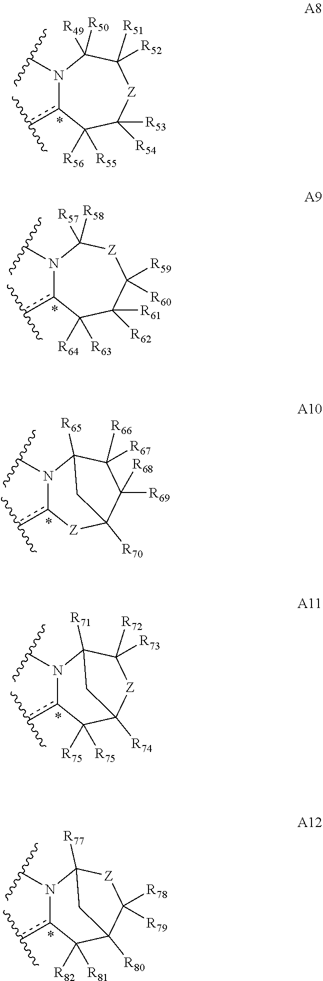

where R₇ to R₈₂ are each independently selected from the group consisting of: hydrogen, an optionally substituted C₁-C₁₀ alkyl, an optionally substituted C₃-C₈ cycloalkyl, an optionally substituted C₂-C₈ alkenyl, an optionally substituted C₁-C₁₀ alkoxy, an optionally substituted C₂-C₈ alkenyloxy, an optionally substituted aryl, an optionally substituted aryloxy, a first optionally substituted heterocycle, an optionally substituted heterocycleoxy, hydroxy, and an optionally substituted amino; and/or one or more of R₇ to R₈₂ form part of an optionally substituted carbocycle or an optionally substituted heterocarbocycle; and/or Z is O or NR₈₃, wherein R₈₃ is hydrogen or C₁-C₁₀ alkyl e.g. e.g. methyl, ethyl, propyl, isopropyl, butyl (e.g. n-butyl, secondary butyl, isobutyl, tertiary butyl), pentyl (e.g. n-pentyl, tert-pentyl, neopentyl, isopentyl, sec-pentyl, 3-pentyl), etc.

In another aspect of the invention, a compound according to formula II, pharmaceutically acceptable salt, or solvate thereof is provided, where R₄ and R₆ are independently substituted with hydrogen or C₁-C₁₀ alkyl, or halogen.

In another aspect of the invention, a compound according to formula II, pharmaceutically acceptable salt, or solvate thereof is provided, where R₃ is hydrogen.

In another aspect of the invention, a compound according to formula II, pharmaceutically acceptable salt, or solvate thereof is provided, where m is 0 or 1 to 3 and at least one R is a halogen e.g. F, Cl or Br.

In another aspect of the invention, a compound according to formula II, pharmaceutically acceptable salt, or solvate thereof is provided, where R₄ and R₆ are independently substituted hydrogen or C1-C10 alkyl, halogen.

In another aspect of the invention, a compound according to formula II, pharmaceutically acceptable salt, or solvate thereof is provided, where R₃ are independently substituted hydrogen or C1-C10 alkyl, halogen.

In another aspect of the invention, a compound according to formula II, pharmaceutically acceptable salt, or solvate thereof is provided, where R₂ is hydrogen.

In another aspect of the invention, a compound according to formula II, pharmaceutically acceptable salt, or solvate thereof is provided, where ring A is represented by A1 and R₇ to R₁₀ are hydrogen.

In another aspect of the invention, a compound according to formula II, pharmaceutically acceptable salt, or solvate thereof is provided, where ring A is represented by A1; one R₇ to R₁₀ is optionally substituted C1-C10 alkyl and the others are hydrogens.

In another aspect of the invention, a compound according to formula II, pharmaceutically acceptable salt, or solvate thereof is provided, where ring A is represented by A1 and Z is oxygen.

In another aspect of the invention, a compound according to formula II, pharmaceutically acceptable salt, or solvate thereof is provided, where ring A is represented by A1 and Z is NR₈₃, wherein R₈₃ is hydrogen or C1-C10 alkyl.

In another aspect of the invention, a compound according to formula II, pharmaceutically acceptable salt, or solvate thereof is provided, where ring A is represented by A2 and R₁₁ to R₁₄ are hydrogen.

In another aspect of the invention, a compound according to formula II, pharmaceutically acceptable salt, or solvate thereof is provided, where ring A is represented by A2; one R₁₁ to R₁₄ is optionally substituted C1-C10 alkyl and the others are hydrogens.

In another aspect of the invention, a compound according to formula II, pharmaceutically acceptable salt, or solvate thereof is provided, where ring A is represented by A2 and Z is oxygen.

In another aspect of the invention, a compound according to formula II, pharmaceutically acceptable salt, or solvate thereof is provided, where ring A is represented by A2 and Z is NR₈₃, wherein R83 is hydrogen or C₁-C₁₀ alkyl.

In another aspect of the invention, a compound according to formula II, pharmaceutically acceptable salt, or solvate thereof is provided, where ring A is represented by A3 and R₁₅ to R₂₀ are hydrogen.

In another aspect of the invention, a compound according to forirrula II, pharmaceutically acceptable salt, or solvate thereof is provided, where ring A is represented by A3; one R₁₅ to R₂₀ is optionally substituted C₁-C₁₀ alkyl and the others are hydrogens.

In another aspect of the invention, a compound according to formula II, pharmaceutically acceptable salt, or solvate thereof is provided, where ring A is represented by A3 and Z is oxygen.

In another aspect of the invention, a compound according to formula II, pharmaceutically acceptable salt, or solvate thereof is provided, where ring A is represented by A3 and Z is NR₈₃, wherein R₈₃ is hydrogen or C₁-C₁₀ alkyl.

In another aspect of the inventio, a compound according to formula II, pharmaceutically acceptable salt, or solvate thereof is provided, where ring A is represented by A4 and R₂₁ to R₂₆ are hydrogen.

In another aspect of the invention, a compound according to formula II, pharmaceutically acceptable salt, or solvate thereof is provided, where ring A is represented by A4; one R₂₁ to 11.7₆ is optionally substituted C 1-C10 alkyl and the others are hydrogens.

In another aspect of the invention, a compound according to formula II, pharmaceutically acceptable salt, or solvate thereof is provided, where ring A is represented by A4 and Z is oxygen.

In another aspect of the invention, a compound according to formula II, pharmaceutically acceptable salt, or solvate thereof is provided, where ring A is represented by A4 and Z is NR₈₃, wherein R₈₃ is hydrogen or C₁-C₁₀ alkyl.

In another aspect of the invention, a compound according to formula II, pharmaceutically acceptable salt, or solvate thereof is provided, where ring A is represented by A5 and R₂₇ to R₃₂ are hydrogen.

In another aspect of the invention, a compound according to formula II, pharmaceutically acceptable salt, or solvate thereof is provided, where ring A is represented by A5; one R₂₇ to R₃₂ is optionally substituted C₁-C₁₀ alkyl and the others are hydrogens.

In another aspect of the invention, a compound according to formula II, pharmaceutically acceptable salt, or solvate thereof is provided, where ring A is represented by A5 and Z is oxygen.

In another aspect of the invention, a compound according to formula II, pharmaceutically acceptable salt, or solvate thereof is provided, where ring A is represented by A5 and Z is NR₈₃, wherein R₈₃ is hydrogen or C₁-C₁₀ alkyl.

In another aspect of the invention, a compound according to formula II, pharmaceutically acceptable salt, or solvate thereof is provided, where ring A is represented by A6 and R₃₃ to R₄₀ are hydrogen.

In another aspect of the invention, a compound according to foiiiiula II, pharmaceutically acceptable salt, or solvate thereof is provided, where ring A is represented by A6; one R₃₃ to R₄₀ is optionally substituted C₁-C₁₀ alkyl and the others are hydrogens.

In another aspect of the invention, a compound according to folinula II, pharmaceutically acceptable salt, or solvate thereof is provided, where ring A is represented by A6 and Z is oxygen.

In another aspect of the invention, a compound according to formula II, pharmaceutically acceptable salt, or solvate thereof is provided, where ring A is represented by A6 and Z is NR₈₃, wherein R₈₃ is hydrogen or C₁-C₁₀ alkyl.

In another aspect of the invention, a compound according to formula II, pharmaceutically acceptable salt, or solvate thereof is provided, where ring A is represented by A7 and R.₄₁ to R₄₈ are hydrogen.

In another aspect of the invention, a compound according to formula II, pharmaceutically acceptable salt, or solvate thereof is provided, where ring A is represented by A7; one R₄₁ to R₄₈ is optionally substituted C₁-C₁₀ alkyl and the others are hydrogens.

In another aspect of the invention, a compound according to formula II, pharmaceutically acceptable salt, or solvate thereof is provided, where ring A is represented by A7 and Z is oxygen.

In another aspect of the invention, a compound according to formula II, pharmaceutically acceptable salt, or solvate thereof is provided, where ring A is represented by A7 and Z is NR₈₃, wherein R₈₃ is hydrogen or C₁-C₁₀ alkyl.

In another aspect of the invention, a compound according to formula II, pharmaceutically acceptable salt, or solvate thereof is provided, where ring A is represented by A8 and R₄₉ to R₅₆ are hydrogen.

In another aspect of the invention, a compound according to formula II, pharmaceutically acceptable salt, or solvate thereof is provided, where ring A is represented by A8; one R₄₉ to R₅₆ is optionally substituted C₁-C₁₀ alkyl and the others are hydrogens.

In another aspect of the ivention, a compound according to formula II, pharmaceutically acceptable salt, or solvate thereof is provided, where ring A is represented by A8 and Z is oxygen.

In another aspect of the invention, a compound according to formula II, pharmaceutically acceptable salt, or solvate thereof is provided, where ring A is represented by A8 and Z is NR₈₃, wherein R83 is hydrogen or C₁-C₁₀ alkyl.

In another aspect of the invention, a compound according to formula II, pharmaceutically acceptable salt, or solvate thereof is provided, where ring A is represented by A9 and R₅₇ to R₆₄ are hydrogen.

In another aspect of the invention, a compound according to formula II, pharmaceutically acceptable salt, or solvate thereof is provided, where ring A is represented by A9; one R₅₉ to R₆₄ is optionally substituted C₁-C₁₀ alkyl and the others are hydrogens.

In another aspect of the invention, a compound according to formula II, pharmaceutically acceptable salt, or solvate thereof is provided, where ring A is represented by A9 and Z is oxygen.

In another aspect of the invention, a compound according to formula II, pharmaceutically acceptable salt, or solvate thereof is provided, where ring A is represented by A9 and Z is NR₈₃, wherein R83 is hydrogen or C₁-C₁₀ alkyl.

In another aspect of the invention, a compound according to formula II, pharmaceutically acceptable salt, or solvate thereof is provided, where ring A is represented by A10 and R₆₅ to R₇₀ are hydrogen.

In another aspect of the invention, a compound according to formula II, pharmaceutically acceptable salt, or solvate thereof is provided, where ring A is represented by A10; one R₆₅ to R₇₀ is optionally substituted C₁-C₁₀ alkyl and the others are hydrogens.

In another aspect of the invention, a compound according to formula II, pharmaceutically acceptable salt, or solvate thereof is provided, where ring A is represented by A10 and Z is oxygen.

In another aspect of the invention, a compound according to formula II, pharmaceutically acceptable salt, or solvate thereof is provided, where ring A is represented by A10 and Z is NR₈₃, wherein R₈₃ is hydrogen or C₁-C₁₀ alkyl.

In another aspect of the invention, a compound according to formula II, pharmaceutically acceptable salt, or solvate thereof is provided, where ring A is represented by A11 and R₇₁ to R₇₆ are hydrogen.

In another aspect of the invention, a compound according to foimula II, pharmaceutically acceptable salt, or solvate thereof is provided, where ring A is represented by A11; one R₇₁ to R₇₆ is optionally substituted C₁-C₁₀ alkyl and the others are hydrogens.

In another aspect of the invention, a compound according to formula II, pharmaceutically acceptable salt, or solvate thereof is provided, where ring A is represented by A11 and Z is oxygen.

In another aspect of the invention, a compound according to formula II, pharmaceutically acceptable salt, or solvate thereof is provided, where ring A is represented by A11 and Z is NR₈₃, wherein R₈₃ is hydrogen or C₁-C₁₀ alkyl.

In another aspect of the invention, a compound according to formula II, pharmaceutically acceptable salt, or solvate thereof is provided, where ring A is represented by A12 and R₇₇ to R₈₇ are hydrogen.

In another aspect of the invention, a compound according to formula II, pharmaceutically acceptable salt, or solvate thereof is provided, where ring A is represented by A12; one R₇₇ to R₈₂ is optionally substituted C₁-C₁₀ alkyl and the others are hydrogens.

In another aspect of the invention, a compound according to formula II, pharmaceutically acceptable salt, or solvate thereof is provided, where ring A is represented by A12 and Z is oxygen.

In another aspect of the invention, a compound according to formula II, pharmaceutically acceptable salt, or solvate thereof is provided, where ring A is represented by A12 and Z is NR₈₃, wherein R₈₃ is hydrogen or C₁-C₁₀ alkyl.

In some embodiments, the compound of formula II has the formula:

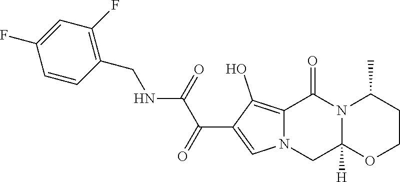 [SMILES: C[C@@H]1CCO[C@H]2Cn3cc(C(=O)C(=O)NCc4ccc(F)cc4F)c(O)c3C(=O)N12]

In some embodiments, the compound of formula II has the formula:

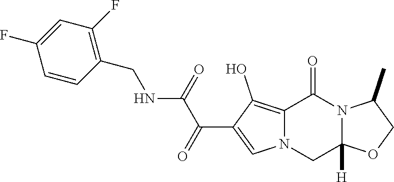 [SMILES: C[C@H]1CO[C@@H]2Cn3cc(C(=O)C(=O)NCc4ccc(F)cc4F)c(O)c3C(=O)N12]

In some embodiments, the compound of formula II has the formula:

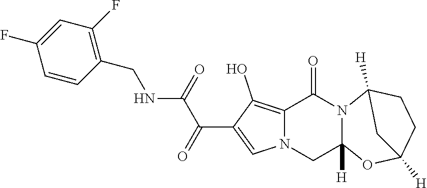 [SMILES: O=C(NCc1ccc(F)cc1F)C(=O)c1cn2c(c1O)C(=O)N1[C@H]3CC[C@H](C3)O[C@@H]1C2]

In some embodiments, the compound of formula II has the formula:

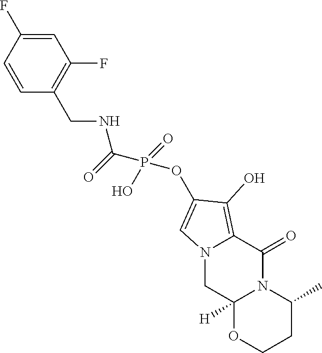 [SMILES: C[C@@H]1CCO[C@H]2Cn3cc(OP(=O)(O)C(=O)NCc4ccc(F)cc4F)c(O)c3C(=O)N12]

In some embodiments, the compound of formula II has the formula:

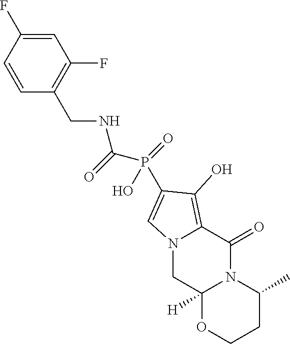 [SMILES: C[C@@H]1CCO[C@H]2Cn3cc(P(=O)(O)C(=O)NCc4ccc(F)cc4F)c(O)c3C(=O)N12]

In some embodiments, the compound of formula II has the formula:

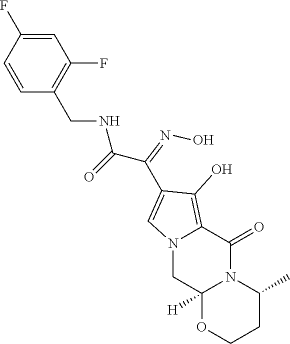 [SMILES: C[C@@H]1CCO[C@H]2Cn3cc(/C(=N\O)C(=O)NCc4ccc(F)cc4F)c(O)c3C(=O)N12]

In some embidiments, the compound of formula II has the formula:

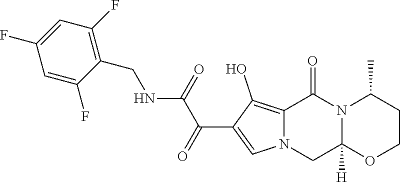 [SMILES: C[C@@H]1CCO[C@H]2Cn3cc(C(=O)C(=O)NCc4c(F)cc(F)cc4F)c(O)c3C(=O)N12]

### Exemplary Preparation of the Compounds

In embodiments of the present invention, the synthesis of the pyrrole and pyridone derivatives relies on the synthesis of the key intermediate 17 of formula:

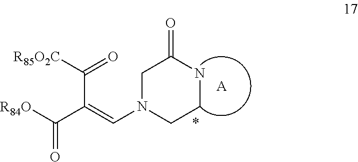

from the compound of formula 10

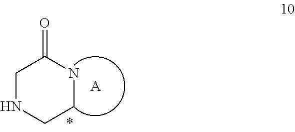

where,

A is an optionally substituted 3-7 carbon heterocycle,

* indicates an R- or S-configuration, and

R₈₄ and R₈₅ are independently hydrogen or C₁-C₁₀ alkyl e.g. methyl, ethyl, propyl, isopropyl, etc.

Compound of formula 10 is prepared as previously disclosed¹⁷ by reacting a suitable glycine ester with a suitable acetaldehyde with palladium on carbon (Pd/C) under hydrogen gas pressure such as e.g. pressure from 30 to 70 bar, such as, 30, 35, 40, 45, 50, 55, 60, 65, 70 bar.

In some aspects of the invention, a generic synthesis of the compound of formula 17 is as follows:

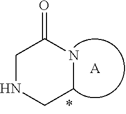

(in which ring A is an optionally substituted heterocycle as defined above) is reacted with a propiolate with generic formula 8,

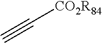

where R₈₄ is an alkyl e.g. methyl, ethyl, propyl, isopropyl, etc., preferably methyl or ethyl; to form compound of formula 18,

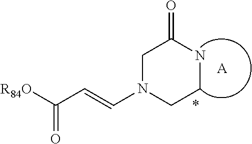

This Michael addition is carried out in a suitable solvent (e.g. THF, DCM, ethyl acetate, acetone, NMP, DMF, MeCN or DMSO) and at suitable temperature such as e.g. in a temperature range of from about 30 to 180° C., such as 30, 40, 50, 60, 70, 80, 90, 100, 110, 120, 130, 140, 150, 160, 170 or 180, e.g. in THF under reflux.

Compound 18 is reacted with an oxalyl chloride of formula 19,

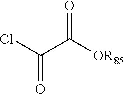

where R₃₅ is an alkyl e.g. methyl, ethyl, propyl, isopropyl, etc, preferably methyl or ethyl, to form compound of formula 17. This reaction is carried out in a suitable base (e.g. DCC, EDC, HATU, DMAP, Et₃N), preferably DMAP, a suitable solvent (e.g. THF, DCM, ethyl acetate, acetone, NMP, DMF, MeCN or DMSO), preferably DCM, and at suitable temperature such as e.g. in a temperature range of from about 15 to 55° C., such as 10,15, 20, 25, 30, 35, 40, 45, 50 or 55° C. and preferably at room temperature.

In another aspect of the invention, the compound of formula 17 is key to the synthesis of the tricyclic core structure. Under basic conditions, the enolate of compound 17 can attack the carbonyls at position C1, C2, or C3 which could lead to three potential cyclized products:

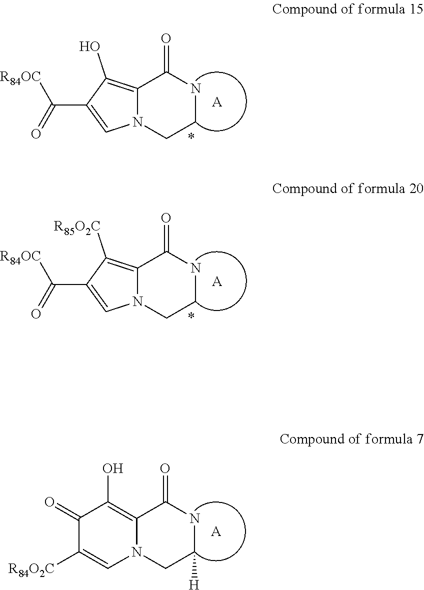

where ring A is an optionally substituted heterocycle as defined above and R₈₄ is an alkyl e.g. methyl, ethyl, propyl, isopropyl, etc., preferably methyl or ethyl.

In further aspects of the invention, the compound of formula 15 is provided by reacting compound of formula 17 with suitable magnesium salt, (e.g. Mg(OMe)₂ or Mg(OtBu)₂, MgBr.OEt₂, MgO, MgClO₄, MgX₂ (where X ═F, Cl, Br, I), preferably Mg(OtBu)₂).The reaction is performed in suitable solvent, examples of which include but not limited to: MeOH, EtOH, ethyl acetate, acetone, NMP, or DMSO, preferably MeOH. This reaction is carried out in at suitable temperature such as e.g. in a temperature range of from about 30 to 140° C., such as 30, 40, 50, 60, 70, 80, 90, 100, 110, 120, 130, 140, 150° C. and preferably at 100° C. Those of skill in the art will recognize that the reaction describe herein may be conducted using or incorporating known reaction strategy, including but not limited to batch synthesis, microwave or flow synthesis, preferably in microwave at 100° C.

In further aspects of the invention, the compound of formula 20 is provided by reacting compound of formula 17 with strong base which include but not limited to: NaH, LDA, LiHMDS, DBU, preferable DBU. The reaction is performed in suitable solvent, examples of which include but not limited to: MeOH, EtOH, THF, DCM, ethyl acetate, acetone, NMP, DMF, MeCN or DMSO, preferably THF. This reaction is carried out in at suitable temperature such as e.g. in a temperature range of from about 20 to 140° C., such as 20, 30, 40, 50, 60, 65, 70, 80, 90, 100, 110, 120, 130, 140, 150° C. and preferably at 65° C.

In further aspects of the invention, the compound of formula 7 is provided by reacting compound of formula 17 with suitable magnesium salt (e.g. Mg(OtBu)₂; MgX₂, where X is Cl, Br or I; MgBr.OEt₂), preferably MgBr.OEt₂, in a suitable base such as DBU, DIPEA, Et₃N, preferably Et₃N. The reaction is performed in suitable solvent, examples of which include but not limited to: MeOH, EtOH, THF, DCM, ethyl acetate, acetone, NMP, DMF, MeCN or DMSO, preferably DCM. This reaction is carried out in at suitable temperature such as e.g. in a temperature range of from about −20 to 30° C., such as −20, −10, 0, 10, 20, 25, 30, 40° C. and preferably at 0-25° C. (room temperature is generally suitable). Those of skill in the art will recognize that the reaction describe herein may be conducted using or incorporating known reaction strategy, including but not limited to batch synthesis, microwave or flow synthesis

In one embodiment, a compound of formula 15 is reacted with a compound of formula 16:

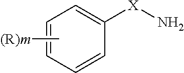

where,

X is selected from the group consisting of a single bond; a heteroatom or a heteroatomic group selected from the group consisting of O, S, SO, SO₂ and NH; C₁-C₆ alkylene; C₁-C₆ heteroalkylene; C₂-C₆ alkenylene; and C₂-C₆ heteroalkenylene;

R is independently selected from halogen and Substituent group SI; wherein substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C₁-C₁₀ alkyl, C₁-C₁₀ alkoxy, a carbamoyl optionally substituted with mono- or di-C₁-C₁₀ alkyl, an optionally substituted C₁-C₁₀ alkyl sulfonyl amino, halogenated C₁-C₁₀ alkoxy and hydroxy C₁-C₁₀ alkyl; and m is 0, 1, 2, or 3,

in two steps which includes,

1. Step 1: reacting compound of formula 15 with a suitable strong base, examples include but are not limited to NaOH, KOH, LDA, LiHMDS, preferably NaOH; in a suitable solvent, examples of which include but not limited to: MeOH, EtOH, THF, DCM, ethyl acetate, acetone, NMP, DMF, MeCN or DMSO, preferably MeOH; in at suitable temperature such as e.g. in a temperature range of from about 20 to 90° C., such as 20, 25, 30, 35, 40, 45, 50, 55, 60, 65, 70, 75, 80, 85, 90 and preferably at 60° C.

2. Step 2: treating the reaction mixture with HATU and base such as e.g. DIPEA, DBU, Et₃N and preferably DIPEA; in a suitable solvent, examples of which include but not limited to: MeOH, EtOH, THF, DCM, ethyl acetate, acetone, NMP, DMF, MeCN or DMSO, preferably MeCN ; in at suitable temperature such as e.g. in a temperature range of from about 0 to 60° C., such as 0, 5, 10, 15, 20, 25, 30, 35, 40, 45, 50, 55, 60° C. and preferably at room temperature.

to form a compound of formula II:

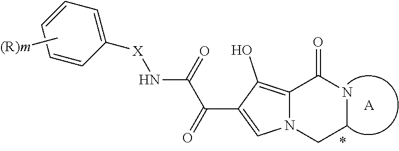

In a variation on the above intermediate of formula 17 can have a modified structure of formula 17-a:

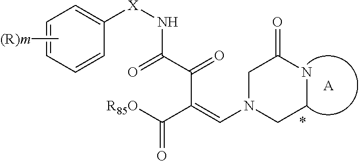

where,

A is an optionally substituted heterocycle or heterocarbocycle;

* indicates an R- or S-configuration,

X is selected from the group consisting of a single bond; a heteroatom or a heteroatomic group selected from the group consisting of O, S, SO, SO₂ and NH; C₁-C₆ alkylene; C₁-C₆ heteroalkylene; C₂-C₆ alkenylene; and C₂-C₆ heteroalkenylene,

R₈₅ are hydrogen or C₁-C₁₀ alkyl,

R is independently selected from halogen and and a substituent group S1;
wherein substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C₁-C₁₀ alkyl, C₁-C₁₀ alkoxy, carbamoyl optionally substituted with mono- or di-C₁-C₁₀ alkyl, optionally substituted C₁-C₁₀ alkyl sulfonyl amino, halogenated C₁-C₁₀ alkoxy and hydroxy C₁-C₁₀ alkyl; and

m is 0, 1,2, or 3.

In one embodiment, a compound of formula 17-a could have inhibitory effect on HIV intergrase and could be used to as a treatment for HIV-AIDS.

In one embodiment, a compound of formula 15 is reacted with a compound of formula 16:

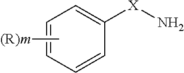

where,

X is selected from the group consisting of a single bond; a heteroatom or a heteroatomic group selected from the group consisting of O, S, SO, SO₂ and NH; C₁-C₆ alkylene; C₁-C₆ heteroalkylene; C₂-C₆ alkenylene; and C₂-C₆ heteroalkenylene;

R is independently selected from halogen and Substituent group Si; wherein substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C₁-C₁₀ alkyl, C₁-C₁₀ alkoxy, a carbamoyl optionally substituted with mono- or di-C₁-C₁₀ alkyl, an optionally substituted C₁-C₁₀ alkyl sulfonyl amino, halogenated C₁-C₁₀ alkoxy and hydroxy C₁-C₁° alkyl; and

m is 0, 1, 2, or 3,

in a suitable solvent, examples of which include but not limited to: MeOH, EtOH, THF, DCM, ethyl acetate, acetone, NMP, DMF, MeCN or DMSO, preferably DMF; in at suitable temperature such as e.g. in a temperature range of from about 30 to 150° C., such as 30, 40, 50, 60, 70, 80, 90, 100, 110, 120, 130, 140, 150° C. and preferably at 90° C. to form a compound of formula:

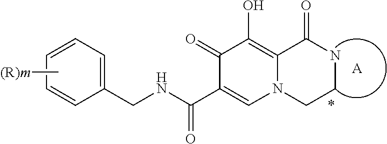

Exemplary synthesis for compound II-a, and dolutegravir Routes 1-3, described in detail below.

### Exemplary Synthesis of Dolutegravir

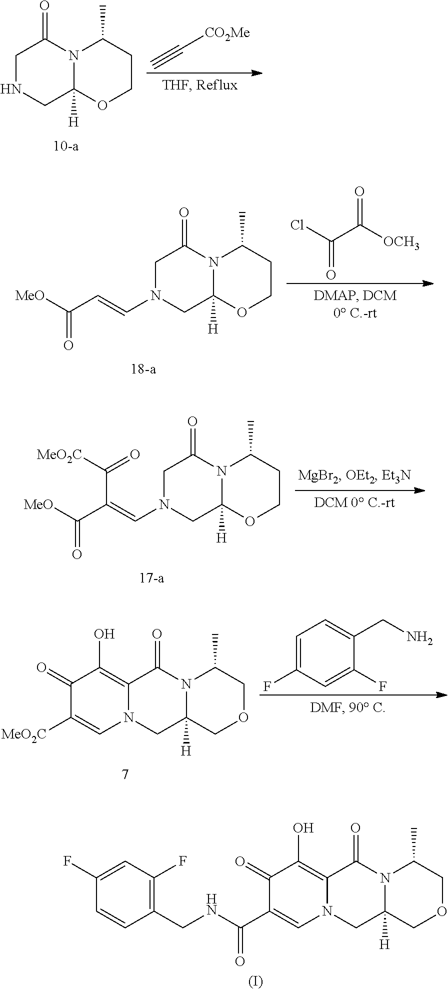

As depicted, the synthetic route 1 comprises four steps with the first being the Michael addition of the methyl propiolate to the bicyclic amine 10-a in to afford 18-a: in a suitable solvent such as but not limited to THF under reflux.

Thereafter, 18-a is reacted with a slightly less electrophilic methyl oxalyl chloride in presence of base such as DMAP and a suitable solvent such as DCM to form compound 17-a. Compound 17-a was reacted with a suitable Mg salt such as but not limited to MgBr₂.OEt₂ base such as but not limited to Et₃N and in a suitable solvent such as DCM to form the tricyclic intermediate 7.

In some aspect, the tricyclic compound 7 in converted dolutegravir by reacting 7 with a 2,4-difluorobenzylamine in a suitable solvent but not limited to DMF, at a temperature range of from about 60° C.-120° C. (e.g. 60, 70, 80, 90, 100, 110 and 120° C.), preferably 90° C. to afford dolutegravir.

### Exemplary Compound 11-a Synthesis

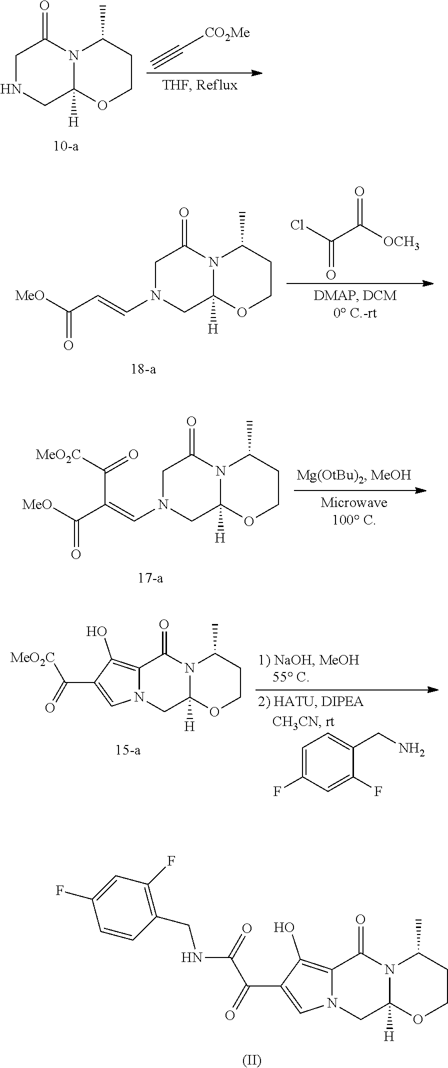

As depicted, the synthetic route 1 comprises four steps with the first being the Michael addition of the methyl propiolate to the bicyclic amine 10-a in to afford 18-a: in a suitable solvent such as but not limited to THF under reflux.

Thereafter, 18-a is reacted with a methyl oxalyl chloride in presence of: a base such as but not limited to DMAP and in a suitable solvent such as DCM to form compound 17-a. Compound 17-a was reacted with a suitable Mg salt such as but not limited to Mg(OtBu)₂ in a suitable solvent such as MeOH to form the tricyclic compound 15-a

In some aspects, the tricyclic 15-a is converted into the final compound II by reacting 15-a with a 2,4-difluorobenzylamine. The chemical transformation begins by treating 15-a with a suitable strong base such as but not limited to NaOH, in a suitable solvent but not limited to MeOH, at a temperature range of from about 30° C.-60° C. (e.g. 30, 35, 40, 45, 50, 55, 60° C.), followed by the addition of a suitable coupling reaction but not limited to HATU and in the presence of a suitable base but not limited to DIPEA in suitable solvent such as but not limited to MeCN at a temperature range of from about 10° C.-40° C. (e.g. room temperature)

### Exemplary Methods of Treatment Using Compounds of Formula II

In some aspects, the compounds of general formula II described herein

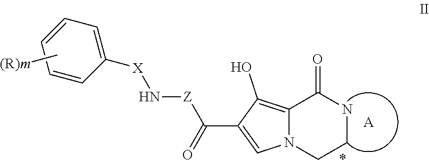

are used to treat viral infections caused by e.g. a retrovirus, in particular a Lentivirus infection such as an infection caused by one or more human immunodeficiency viruses (HIV). Generally, two species of HIV cause HIV infection and over time acquired immunodeficiency syndrome (AIDS). Any HIV infection may be treated at any stage of the disease using the compounds provided herein, e.g. at an early stage when virus is detectable by overt symptoms are not present, or at a later stage such as full blown AIDS.

RNA viral infections caused by one or more members of the Orthomyxoviridae family of RNA viruses may also be treated. In particular, the genera Influenzavirus A, Influenzavirus B, and Influenzavirus C, which cause infections in humans. More particularly, Influenzavirus A viral infections are treated, i.e. those which cause flu pandemics.

In some aspects, the compound used is the compound II-a of formula

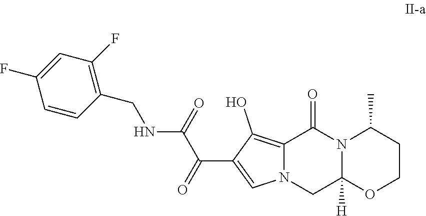 [SMILES: C[C@@H]1CCO[C@H]2Cn3cc(C(=O)C(=O)NCc4ccc(F)cc4F)c(O)c3C(=O)N12]

In some aspect, CEM-SS cells were incubated at 37° C./5% CO₂ with serially diluted compound and a known titer of HIV-1_{IIIB} for 6 days. The CEM-SS cell line is a derivative of the human T lymphosarcoma cell line CEM that is permissive for vif-deficient HIV-1 replication. Following the incubation, the cells were stained with the tetrazolium dye XTT and read at 460/650 nm on a spectrophotometer to evaluate cellular viability. Efficacy and toxicity values were calculated using linear regression analysis. The efficacy of compound H-a was compared to that of dolutegravir. The EC50 of compound II-a was in a range of 0.070-0.02 nM (e.g. 0.070, 0.075, 0.080, 0.085, 0.090, 0.095, 0.010, 0.015 or 0.02 nM)

| Compound | EC₅₀ | |
|---|---|---|
| Control: Azidothymidine (AZT) | 0.005 | μM |
| Compound I: Dolutegravir (DTG) | 0.12 | nM |
| Compound II-a | 0.095 | nM |

Exemplary Compositions Comprising the Compound II and/or Derivatives and Methods of Administering the Compound II or Derivative Compositions

Provided herein are compositions comprising at least one compound II as described herein, and methods of administering the same to treat e.g. HIV, influenza, etc. Implementation of the methods generally involves identifying patients suffering from or at risk of developing a disease or condition described herein (for example HIV), and administering a composition as described herein by an appropriate route. The exact dosage to be administered may vary depending on the age, gender, weight and overall health status of the individual patient, or on other treatments being received by the patient, as well as the extent or progression of the disease condition being treated and the precise etiology of the disease. However, in general for administration to mammals (e.g. humans), sufficient composition is administered to achieve compound II or derivative dosages in the range of from about 0.1 to about 60 mg or more per kg of body weight per 24 hr., and preferably about 0.1 to about 30 mg of compound II or derivative per kg of body weight per 24 hr., and more preferably about 0.1 to about 10 mg of compound II or derivative per kg of body weight per 24 hr. are effective. Accordingly, daily doses (in terms of compound II or derivative) generally range from about 6 milligram to about 3600 milligrams per person per day. In some aspects, the dose is from about 10 milligrams to about 2000 milligrams per person per day, or about 100 milligrams to about 1000 milligrams per person per day. The dose will vary with the route of administration, the bioavailability, and the particular formulation that is administered, as well as according to the nature of the malady that is being prevented or treated.

The compositions are generally administered in a pharmaceutically acceptable formulation which includes suitable excipients, elixirs, binders, and the like (generally referred to as “pharmaceutically and physiologically acceptable carriers”), which are pharmaceutically acceptable and compatible with the active ingredients. The compound II or derivatives may be present in the formulation as pharmaceutically acceptable salts (e.g. alkali metal salts such as sodium, potassium, calcium or lithium salts, ammonium, etc.) or as other complexes. It should be understood that the pharmaceutically acceptable formulations include solid, semi-solid, and liquid materials conventionally utilized to prepare solid, semi-solid and liquid dosage forms such as tablets, capsules, liquids, aerosolized dosage forms, and various injectable forms (e.g. forms for intravenous administration), etc. Suitable pharmaceutical carriers include but are not limited to inert solid diluents or fillers, sterile aqueous solutions and various organic solvents. Examples of solid carriers (diluents, excipients) include lactose, starch, conventional disintegrating agents, coatings, lactose, terra alba, sucrose, talc, gelatin, agar, pectin, acacia, magnesium stearate, stearic acid and lower alkyl ethers of cellulose. Examples of liquid carriers include but are not limited to various aqueous or oil based vehicles, saline, dextrose, glycerol, ethanol, isopropanol, phosphate buffer, syrup, peanut oil, olive oil, phospholipids, fatty acids, fatty acid amines, polyoxyethylene, isopropyl myristate, ethyl cocoate, octyl cocoate, polyoxyethylenated hydrogenated castor oil, paraffin, liquid paraffin, propylene glycol, celluloses, parabens, stearyl alcohol, polyethylene glycol, isopropyl myristate, phenoxyethanol, and the like, or combinations thereof. Water may be used as the carrier for the preparation of compositions which may also include conventional buffers and agents to render the composition isotonic. Oral dosage forms may include various thickeners, flavorings, diluents, emulsifiers, dispersing aids, binders, coatings and the like. The composition of the present disclosure may contain any such additional ingredients so as to provide the composition in a form suitable for the intended route of administration. In addition, the composition may contain minor amounts of auxiliary substances such as wetting or emulsifying agents, pH buffering agents, and the like. Similarly, the carrier or diluent may include any sustained release material known in the art, such as glycerol monostearate or glycerol distearate, alone or mixed with wax. Other potential additives and other materials (preferably those which are generally regarded as safe [GRAS]) include: colorants; flavorings; surfactants (TWEEN®, oleic acid, etc.); and solvents, stabilizers, binders or encapsulants (lactose, liposomes, etc.). Preservatives such as methyl paraben or benzalkium chloride may also be used. Depending on the formulation, it is expected that the active components (e.g. at least one compound II or derivative) will be present at about 1% to about 99% of the composition and the vehicular “carrier” will constitute about 1% to about 99% of the composition. The pharmaceutical compositions of the present disclosure may include any suitable pharmaceutically acceptable additives or adjuncts to the extent that they do not hinder or interfere with the therapeutic effect(s) of the composition. Still other suitable formulations for use in the present disclosure can be found, for example in Remington's Pharmaceutical Sciences 22nd edition, Allen, Loyd V., Jr editor (September 2012); and Akers, Michael J. Sterile Drug Products: Formulation, Packaging, Manufacturing and Quality; publisher Informa Healthcare (2010).

The compositions (preparations) of the present disclosure are formulated for administration by any of the many suitable means which are known to those of skill in the art, including but not limited to: orally, by injection, rectally, by inhalation, intravaginally, intranasally, topically, as eye drops, via sprays, transdermally, sublingually, by rectal and buccal delivery, by inhalation of an aerosol, by microneedle delivery, etc. In some aspects, the mode of administration is oral, by injection or intravenously.

The administration of the compound of the present disclosure may be intermittent, or at a gradual or continuous, constant or controlled rate (e.g. in a sustained release formulation which further extends the time of bioavailability, or IV). In addition, the time of day and the number of times per day that the pharmaceutical formulation is administered may vary and are best determined by a skilled practitioner such as a physician.

Administration of the compound by any means may be carried out as a single mode of therapy, or in conjunction with one or more additional agents or therapies and treatment modalities, e.g. antibiotics, pain medication, vaccinations, in a “cocktail” with other anti-viral agents such as those typically used to treat HIV; or with anti-nausea and/or pain medications, fluids, anti-diarrheals, etc. for subject with influenza. All such combinations are encompassed herein. “In conjunction with” refers to both administration of a separate preparation of the one or more additional agents, and also to inclusion of the one or more additional agents in a composition of the present disclosure.

The subject to whom the composition is administered is generally a mammal, frequently a human, but this is not always the case. Veterinary applications of this technology are also contemplated, e.g. for companion pets (cats, dogs, etc.), or for livestock and faun animals, for pigs, cattle, etc., and even for “wild” animals that have special value or that are under the care of a veterinarian, e.g. animals in preserves or zoos, injured animals that are being rehabilitated, etc.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting.

Where a range of values is provided, it is understood that each intervening value between the upper and lower limit of that range (to e.g. a tenth of the unit) is included in the range and encompassed within the invention, unless the context or description clearly dictates otherwise. In addition, smaller ranges between any two values in the range are encompassed, unless the context or description clearly indicates otherwise.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which maybe readily separated from or combined with the features of any of the other several embodiments without departing from the scope or spirit of the present invention. Any recited method can be carried out in the order of events recited or in any other order which is logically possible.

### EXAMPLES

Examples 1-6 show the synthesis steps to produce 17, a key intermediate in the synthetic pathways disclosed herein.

Materials. Glycine methyl ester hydrochloride (99%), palladium on activated carbon (10% Pd, unreduced, dry), anhydrous methanol (MeOH), tetrahydrofuran, and acetic acid (AcOH) were purchased from Fisher Scientific. Triethylamine (Et₃N), 2,2-dimethoxyacetaldehyde (60 wt.% in H₂O), anhydrous dichloromethane (99.9%), anhydrous toluene (99.8%), N, N-diisopropylethylamine (>99%), 2,4-difluorobenzylamine (98%), and magnesium di-tert butoxide (≥85%) were purchased from Sigma Aldrich. Sodium bicarbonate, sodium hydroxide, and trifluoroacetic acid were purchased from VWR. (R)-3-aminobutan-1-ol and methyl oxalyl chloride were purchased from Combi-Blocks. Methyl propiolate and ethyl acetate were purchased from Alfa Aesar. HATU was purchased from Chempep Inc. Techniques. ¹H NMR (400 MHz) and ¹³C NMR (100 MHz) spectra were recorded on a Bruker Biospin NMR spectrometer. Peak multiplicities are denoted as follows: s=singlet, d=double, t=triplet, pent=pentet, hept =heptet, and m =multiplet. Thin layer chromatography (TLC) was performed using Whatman silica gel 60 A plates (250 vim) with fluorescent indicator and visualized using UV lamp (254 nm) or KMnO₄ stain. Flash chromatography was performed on Biotage Isolera™ Spektra Systems with ACI™ and Assist using disposable silica gel columns (60 Å, 40-63 μm particle size). Preparative HPLC was performed using Agilent 1260 Binary Pump comes with 1260 VWD (254 nm and 210 nm).

### Example 1

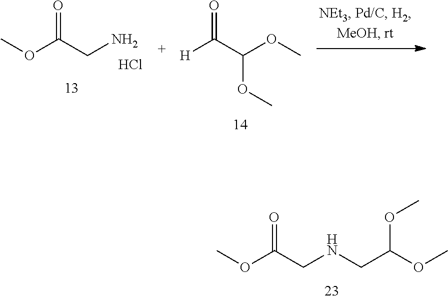

Glycine methyl ester (13) (20.0 g, 0.159 mol) was added at room temperature to a reaction vessel with 100 mL of MeOH. The reaction mixture was mixed until all the glycine methyl ester had dissolved. After mixing, Et₃N (22.2 mL, 0.159 mol) and 2,2-dimethyloxoacetaldehyde (14) (27.8 mL, 0.159 mmol) were added to the reaction vessel at room temperature. Under N₂, Pd/C was transferred to a reaction vessel. An additional 20 mL of MeOH was added. The reaction vessel was kept under H2 (25 bar) for 10-12 hours and the reaction was monitored via TLC. After complete consumption of starting materials, the reaction mixture was filtered through celite to remove the Pd/C. The celite cake was washed with MeOH (200 mL). The reaction mixture was then transferred to a round-bottom flask (RBF) and the methanol was removed under reduced pressure. The mixture was transferred to a separatory funnel and extracted five times with EtOAc (100 mL). The organic layer was dried over anhydrous sodium sulfate and the solvent was removed under reduced pressure. Extraction of the material yielded 23 as a pale-yellow material (26.2 g, 93%).

### Example 2

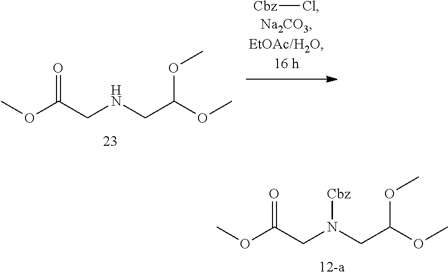

Compound of formula 23 (40.5 g, 0.237 mol) and 500 mL of EtOAc and H₂O mixture were transferred in to three-neck (RBF). The reaction mixture was cooled to 0° C. Na₂CO₃ (75.3 g, 0.710 mol) was added to the reaction mixture while stirring at 0° C. for 10 min. The reaction mixture was left to stir at room temperature overnight. The reaction was monitored by TLC. Following the consumption of 23, the reaction mixture was cooled to 0° C. A solution of 1 M HCl (250 mL) was added to the reaction mixture and stirred for 10 min. The mixture was transferred to a separatory funnel. The layers were separated, and the aqueous layer was extracted four times with EtOAc (200 mL). The organic layers were combined and washed with saturated NaCl (aq) solution (250 mL). The mixture was dried over anhydrous Na₂SO₄. Volatiles were removed under reduced pressure. The resulting residue was purified by flash column chromatography (SiO2, EtOAc/hexanes 80:20 to 50:50) and yielded 12-a as a colorless liquid (60.9 g, 83%).

### Example 3

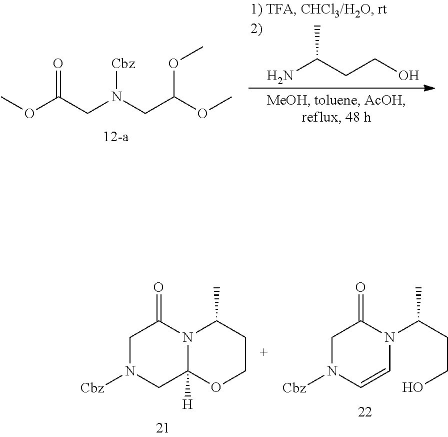

Step 1.

Compound 12-a (25.0 g, 0.08 mol) was transferred into a one-liter round bottom flask and a 1:1 solution of CHCl₃/H₂O (236 mL) was added. The reaction mixture was cooled to 0° C. TFA (123.0 mL, 1.60 mol) was added dropwise over 30 min. The reaction was monitored by TLC. Following the consumption of 12-a, the reaction mixture was transferred to a separatory funnel. The reaction mixture was washed with 10% K₂CO₃ aqueous solution (200 mL). The aqueous layer was extracted four times with DCM (150 mL) and washed with saturated NaCl (aq) solution (250 mL). The mixture was dried over anhydrous Na₂SO₄. Volatiles were removed under reduced pressure. The residue was dried under high vacuum for one hour. A colorless oil (18.0 g, 85%) was obtained as the step one product.

Step 2.

The Step one product (18.0 g, 0.068 mol) and toluene (180 mL) were transferred into a 250 mL round-bottom flask at room temperature. Methanol (7.8 mL), AcOH (3.7 mL) and (R)-3-aminobutan-l-ol (7.40 mL, 0.077 mmol) were added. The reaction mixture was stirred at reflux for 48 h. The reaction was monitored via LC-MS and TLC. Following the consumption of the step one product, volatiles were removed under reduced pressure. The mixture was dissolved in DCM (200 mL) and transferred to a separatory funnel. The reaction mixture was washed with saturated NaHCO₃ solution (25 mL). The aqueous layer was extracted three times with DCM (50 mL). The organic layers were combined and washed with saturated NaCl (aq) solution (50 mL). The resulting mixture was dried over anhydrous Na₂SO₄. Volatiles were removed under reduced pressure. The residue was purified by flash chromatography to yield 21 (14.0 g, 0.046 mol, 71%) and 22 (3.24 g, 0.011 mol, 12%). 22 is not reacted further.

### Example 4

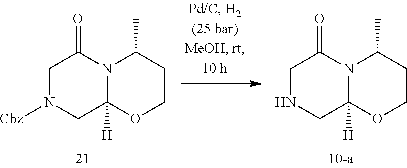 [SMILES: C[C@@H]1CCO[C@H]2CN(C(=O)OCc3ccccc3)CC(=O)N12 ; C[C@@H]1CCO[C@H]2CNCC(=O)N12]

Compound 21 was dissolved in MeOH (90 mL) and transferred to a reaction vessel. Under N₂, Pd/C was transferred to a reaction vessel to which MeOH (10 mL) was then added. The reaction vessel was kept under H₂ (25 bar) for 10 h. The reaction was monitored via TLC. Following the consumption of 21, the reaction mixture was filtered through a bed of celite to remove the Pd/C. The celite cake was washed with MeOH (100 mL). The volatiles were removed under reduced pressure. The resulting residue was kept under high vacuum for an hour. 10-a was obtained as colorless oil (7.36 g, 0.043 mol, 94%) and used in the next step without further purification.

### Example 5

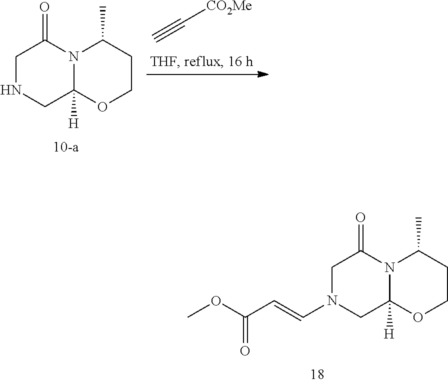

A solution of 10-a (5.33 g, 0.031 mol) and THF (50 mL) was dissolved in 100 mL round-bottom flask. Methyl propiolate (2.63 g, 0.031 mol) was added to the reaction mixture. The reaction was stirred at reflux overnight. The reaction was monitored by TLC. Following the consumption of 10-a, volatiles were removed under reduced pressure. The residue was purified by flash column chromatography and yielded a pale yellow solid, 18-a, (7.27 g, 0.029 mol, 92%). In some cases, material 18-a can be purified via recrystallization using a 4:1 solution of EtOAc/DCM. ¹H NMR (CDCl₃, ppm): 7.34 (d, 1H), 4.95 (m, 2H), 4.64 (d, 1H), 3.92 (m, 2H), 3.81 (m, 2H), 3.65 (overlapping m, 3H), 3.34 (dd, 1H), 2.11 (m, 1H), 1.44 (d, 1H), 1.27 (d, 3H). ^{I3}C NMR (CDCl1₃, ppm): 169.2, 163.6, 150.1, 87.9, 77.7, 62.6, 50.8, 42.6, 29.4, 15.8.

### Example 6

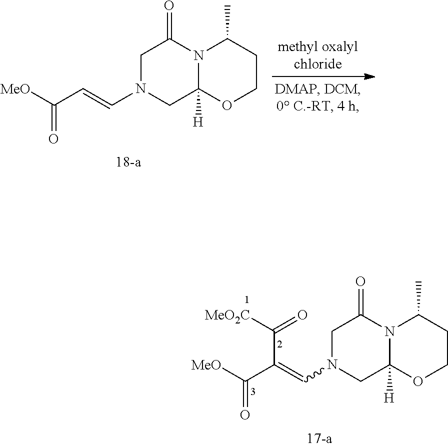 [SMILES: COC(=O)/C=C/N1CC(=O)N2[C@H](C)CCO[C@H]2C1 ; COC(=O)C(=O)C(=CN1CC(=O)N2[C@H](C)CCO[C@H]2C1)C(=O)OC]

Under N₂, a solution of 18-a (0.77 g, 3.0 mmol) and DCM (15 mL) was dissolved in a 25 mL round-bottom flask. The reaction mixture was cooled to 0° C. DMAP (0.4 g, 3.15 mmol) was added at 0° C. and stirred for 5 min. Dropwise, methyl oxalyl chloride was added to the mixture. Ice-bath was removed, and the reaction was warmed to room temperature. The reaction mixture was heated to reflux and stirred at reflux for 4 h. Reaction was monitored by TLC and LC-MS. Following the consumption of 18-a, volatiles were removed under reduced pressure. The residue was purified by flash column and yielded 17-a as a pale-yellow gel (0.99 g, 0.29 mmol, 97%).¹H NMR (MeOD₄, ppm): 7.98 (s, 1H), 5.21 (s, 1H), 4.87 (t, 1H), 4.13 (m, 4H), 4.07 (m, 2H), 3.82 (s, 3H), 3.71 (s, 3H), 3.06 (m, 1H), 1.51 (d, 1H), 1.33 (d, 3H). ¹³C NMR (MeOD₄, ppm): 183.6, 166.5, 163.3, 158.6, 97.4, 77.1, 62.8, 60.2, 55.6, 53.6, 51.5, 50.7, 43.9, 29.2, 14.5.

The reactions described in Examples 7, 8 and 9 below each use 18-a as the starting material. However, reaction conditions are different for each of the three Examples and three different products, 7, 15-a and 20-a, are obtained. Each of the three products is then further reacted (see Examples 10-12) to produce compounds I, II and III.

### Example 7

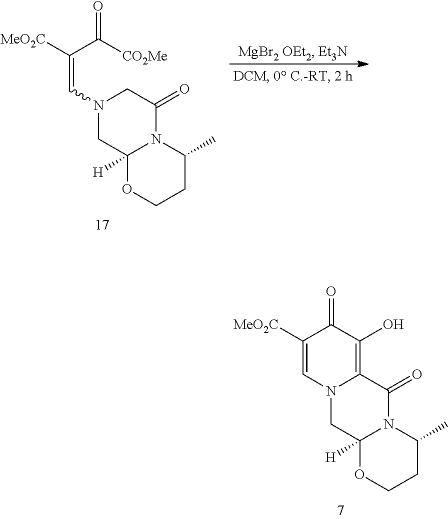

Under N₂, a solution of 17-a (0.34 g, 1.0 mmol) and DCM (12 mL) was dissolved in a 25 mL round-bottom flask. The mixture was cooled to 0° C. MgBr₂OEt₂ (0.32 g, 1.25 mmol) was added at 0° C. and stirred for 15 min. The resulting mixture was observed to be a yellow slurry. Dropwise, Et₃N was added to the reaction mixture. The ice-bath was removed, and the reaction was warmed to room temperature. The mixture was observed to be a brown-red slurry and resulted in a hazy solution overtime. The reaction stirred at room temperature for 2 h. Reaction was monitored by LC-MS. Following the consumption of 17-a, volatiles were removed under reduced pressure. The residue was dissolved in saturated NaHCO₃ (10 mL). The mixture was extracted with DCM to remove the undesired products. The pH of the aqueous layer was adjusted to 1 using 2 M HCl at 0° C. The layers were separated three times with DCM. Product was still observed in aqueous layer. Organic layers were separated and dried over anhydrous Na₂SO₄. Volatiles were removed under reduced pressure and product was dried under high vacuum. 7 was obtained as a white solid (0.21 g, 65%). ¹H NMR (CDCl₃, ppm): 12.47 (broad s, 1 H), 7.91 (s, 1H), 5.40 (q, 1 H), 4.90 (t, 1 H), 4.35 (q, 1H), 4.03 (q, 3H), 3.98 (s, 3H), 2.20 (m, 1 H), 1.55 (d, 1H), 1.53 (d, 3H). ¹³C NMR (CDCl₃, ppm): 169.3, 164.7, 162.4, 156.2, 141.7, 115.3, 114.3, 76.4, 62.6, 52.4, 52.0, 44.7, 29.4, 15.5.

### Example 8

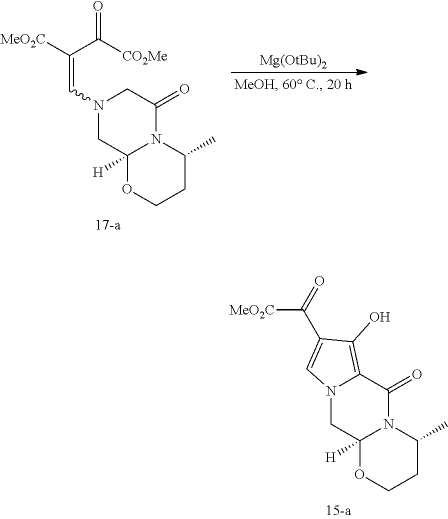

A solution of 17-a (0.200 mg, 0.60 mmol) and anhydrous MeOH (0.78 mL) was dissolved in a 10 mL vial. The reaction was heated to reflux at 60° C. Reaction was monitored by LC-MS. Following the consumption of starting material, the crude mixture was filtered through a syringe filter with 1:1 mixture of acetonitrile and water. The mixture was purified through preparative HPLC to yield 15. ¹H NMR (CDCl₃, ppm): 7.07 (s, 1H), 5.41 (s, 1H), 5.01 (t, 11-1), 4.43 (dd, 1H), 4.15 (d, 1H), 4.05 (overlapping m, 2H), 3.85 (s, 3H), 2.22 (m, 1H), 1.57 (d, 1H), 1.44 (d, 3H). ¹³C NMR (CDCl₃, ppm): 164.7, 161.3, 159.2, 125.4, 122.1, 121.1, 120.8, 78.0, 62.9, 52.5, 47.3, 44.9, 29.4, 15.6.

### Example 9

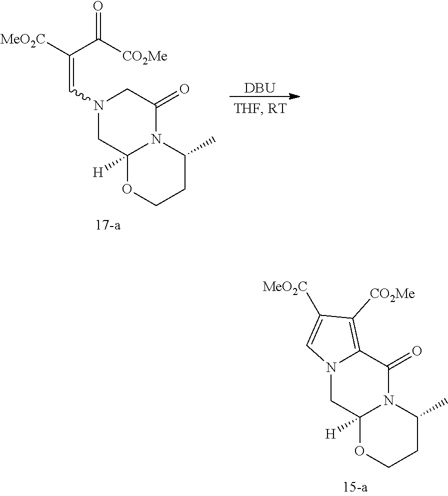

A solution of 17-a 1.0 g, 2.94 mmol) and THF (20 mL) was dissolved in a round-bottom flask and left to stir overnight at room temperature. Reaction was monitored via LC-MS. Following the consumption of 17-a, all volatiles were removed under reduced pressure. The mixture was dissolved in DCM (20 mL) and washed with H₂O (5 mL). The aqueous layer was extracted with DCM (20 mL). The organic layer was washed with 1 M HCl (4 mL). The organic layers were combined and washed with saturated NaCl (aq) solution (50 mL). The resulting mixture was dried over anhydrous Na₂SO₄. Volatiles were removed under reduced pressure. The resulting residue was purified by flash column chromatography (SiO₂, MeOH/DCM 10:1) to yield 15-a (0.90 g, 94%). ¹H NMR (CDCl₃, ppm): 7.19 (s, 1H), 5.20 (q, 1H), 4.93 (t, 1H), 4.20 (dd, 1H), 4.06 (dd, 1 H), 3.91 (overlapping m, 5H), 3.73 (s, 3H), 2.11 (m, 1H), 1.37 (d, 1H), 1.28 (d, 3H). ¹³C NMR (CDCl₃, ppm): 165.9, 162.9. 156.5, 126.1, 122.2, 121.6, 115.6, 78.1, 63.1, 52.9, 51.8, 47.7, 43.6, 29.3, 15.6

### Examples 10-11 Describe the Synthesis of Compounds I, II

### Example 10

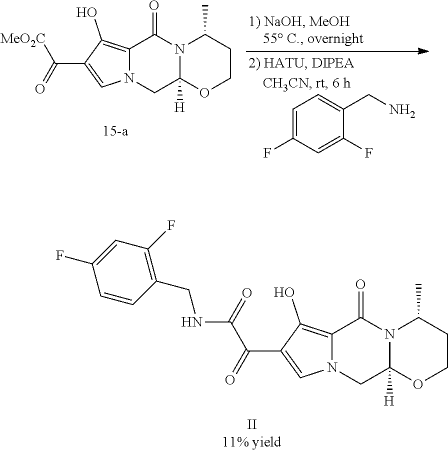 [SMILES: COC(=O)C(=O)c1cn2c(c1O)C(=O)N1[C@H](C)CCO[C@H]1C2 ; NCc1ccc(F)cc1F ; C[C@@H]1CCO[C@H]2Cn3cc(C(=O)C(=O)NCc4ccc(F)cc4F)c(O)c3C(=O)N12]

Methanol (3.2 mL) was added to a RBF with compound 15-a (0.17 g, 0.55 mmol) while stirring. NaOH (0.1170 g, 2.925 mmol) was added to the reaction mixture. The reaction mixture was heated to 55° C. for 13 hours and monitored by LC-MS for the consumption of starting material. Additional NaOH (0.12403 g, 3.1008 mmol) was added to the reaction mixture while stirring at 55° C. for 4 hours. The volatiles were removed under pressure from the crude and the remaining material was acidified to pH 1 with 2 M HCl (aq.) solution and transferred to a separatory funnel. The aqueous solution was extracted with CH₂Cl₂ (8×10 mL). The combined organic layers were dried over anhydrous Na₂CO₃ and the volatiles were removed under reduced pressure to form 0.15 g yellow solids. Methanol was added to the crude (0.15 g) and sonicated, followed by the addition of water. The precipitates that formed were filtered via gravity filtration, dried and characterized (0.08417 g pure II intermediate). Acetonitrile (0.92 mL) and DIPEA (0.16 mL, 0.92 mmol) was added to the filtered solids (0.08417 g) while stirring under N₂ atmosphere at room temperature for 1 hour. HATU (0.27135 mg, 0.71363 mmol) was added to the reaction mixture and stirred at room temperature under N₂ atmosphere for 1 hour. 2,4-difluorobenzylamine (61 μl, 0.51 mmol) was added to the reaction mixture and left to stir for 5 hours while monitoring by LC-MS. Water (0.92 mL) was added to the reaction mixture followed by acidification with 1 M FIC1 (aq.) solution to pH 1. The solution was transferred to a separatory funnel and extracted with EtOAc (2 x 3 mL). The combined organic layers were extracted with saturated NaHCO₃ (1×0.3 mL), and with water (1×0.9 mL). The volatiles were removed under reduced pressure forming II solids (0.53476 g). The solids were purified by prep HPLC (95:5 to 5:95 H₂O/ACN) to yield pure II (0.01265 g, 0.03016 mmol) in 11% yield. ¹H NMR (DMSO-d6, ppm): 12.44 (s, 1H), 10.29 (t, 1H), 8.43 (s, 1H), 7.32 (q, 1H), 7.18 (dt, 111), 7.01 (dt, 1H), 5.38 (t, 1H), 4.73 (p, 1H), 4.48 (overlapping m, 3H), 4.28 (dd, 1H),3.95 (dt, 1H), 3.83 (m, 1H), 1.94 (m, 1H), 1.49 (dd, 1H), 1.26 (d, 3H). ^{I3}C NMR (DMSO-d6, ppm): 170.9, 164.2, 161.4, 161.1, 159.8, 141.1, 131.2, 122.9, 122.8, 117.2, 111.9, 111.7, 104.3, 76.6, 62.5, 51.7, 45.2, 36.2, 29.6, 15.7.

### Example 11

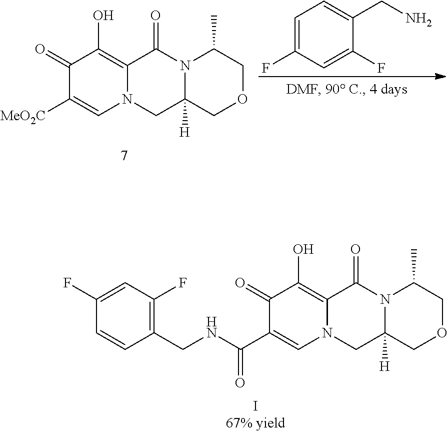

DMF (0.50 mL) was added to a vial with compound 7 (0.15625 g, 0.50683 mmol) while stirring under N₂ atmosphere. 2,4-difluorobenzylmine (0.09 mL, 0.8 mmol) was added to the reaction mixture. The reaction mixture was heated to 90° C. for 4 days and monitored by LC-MS for the consumption of starting material. The reaction mixture was acidified to pH 1 with 2M HCl (aq.) solution and transferred to a separatory funnel. The solution was extracted with CH₂Cl₂ (3×5 mL). The volatiles were removed under reduced pressure. The crude oil (0.30 g) were purified by flash column chromatography (50-100% EtOAc/hexanes and 0-20% MeOH/CH₂Cl₂) I (0.14214 g, 0.33893 mmol) in 67% yield. ^{I}H NMR (DMSO-d6, ppm): 12.44 (s, 1H), 10.75 (s, 1H), 7.88 (s, 1H), 7.37 (q, 1H), 7.21 (dt, 1H), 7.04 (dt, 1H), 5.17 (t, 1H), 4.81 (p, 1H), 4.52 (d, 2H), 4.32 (d, 1H), 4.17 (dd, 1H), 3.98 (t, 1H), 3.82 (dd, 1H), 1.88 (m, 1H), 1.38 (d, 1H), 1.26 (d, 3H).

### Example 12

### In Vitro Testing of Exemplary Compounds I and II Assay was Conducted at ImQuest Biosciences

CEM-SS cells were incubated at 37° C./5% CO₂ with serially diluted compound and a known titer of HIV-1_{IIIB} for 6 days. The CEM-SS cell line is a derivative of the human T lymphosarcoma cell line CEM that is permissive for vif-deficient HIV-1 replication. Following the incubation, the cells were stained with the tetrazolium dye XTT and read at 460/650 nm on a spectrophotometer to evaluate cellular viability. Efficacy and toxicity values were calculated using linear regression analysis.

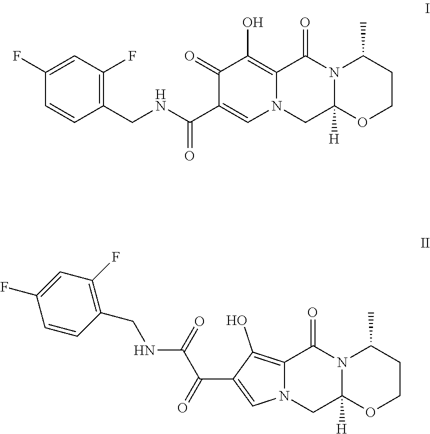 [SMILES: C[C@@H]1CCO[C@H]2Cn3cc(C(=O)NCc4ccc(F)cc4F)c(=O)c(O)c3C(=O)N12 ; C[C@@H]1CCO[C@H]2Cn3cc(C(=O)C(=O)NCc4ccc(F)cc4F)c(O)c3C(=O)N12]

| Compound | EC₅₀ | |
|---|---|---|
| Control: Azidothymidine (AZT) | 0.005 | μM |
| Compound I: Dolutegravir (DTG) | 0.12 | nM |
| Compound II | 0.095 | nM |

### REFERENCES

(1) HIV.gov. Global statistics www.hiv.gov/hiv-basics/overview/data-and-trends/global-statistics (accessed Mar. 26, 2019).

(2) UNAIDS. Global HIV and AIDS statistics-2018 fact sheet. www.unaids.org/en/resources/fact-sheet (accessed Mar. 26, 2019).

(3) Llibre, J. M.; Pulido, F.; Garcia, F.; Deltoro, M. G.; Blanco, J. L.; Delgado, R. Genetic Barrier to Resistance for Dolutegravir. AIDS Rev. 2014, 17 (1), 59-68.

(4) Akihisa, E.; Kawauchi-Miki, S.; Miki, S.; Sato, A.; Wakasa-Morimoto, C.; Fujiwara, T.; Nakahara, K.; Seki, T.; Suyama-Kagitani, A.; Underwood, M. R.; et al. Effects of Raltegravir or Elvitegravir Resistance Signature Mutations on the Barrier to Dolutegravir Resistance In Vitro . Antimicrob. Agents Chemother 2015, 59 (5), 2596-2606. doi.org/10.1128/aac.04844-14.

(5) Wang, H.; Kowalski, M. D.; Lakdawala, A. S.; Vogt, F. G.; Wu, L. An Efficient and Highly Diastereoselective Synthesis of GSK1265744, a Potent HIV Integrase Inhibitor. Org. Lett. 2015, 17 (3), 564-567. doi.org/10.1021/o1503580t.

(6) Wang, H, Goodman SV, Mann D, K. M. The Process for Preparing Carbamoylpyridone Derivatives and Intermediates WO2011/119566. WO2011/119566 A1, 2011.

(7) Sankareswaran, S.; Mannam, M.; Chakka, V.; Mandapati, S. R.; Kumar, P. Identification and Control of Critical Process Impurities: An Improved Process for the Preparation of Dolutegravir Sodium. Org. Process Res. Dev. 2016, 20 (8), 1461-1468. doi.org/10.102/acs.oprd.6b00156.

(8) Sumino, Y.; Masui, M.; Yamada, D.; Ikarashi, F.; Okamoto, K. Method of Producing Compounds Having HIV Integrase Inhibitory Activity. WO 2012/018065,2012.

(9) Ziegler, R. E.; Gupton, B. F.; Roper, T. D.; Jamison, T. F.; Jee, J.-A.; Desai, B. K. 7-Step Flow Synthesis of the HIV Integrase Inhibitor Dolutegravir. Angew Chemie Int. Ed 2018, 57 (24), 7181-7185. doi.org/10.1002/anie.201802256.

(10) Vellanki, S. P.; Nadella, M.; Bhalme, M.; Ramabhotla, R. S.; Arumalla, S. R.; Kilaru, R. B. Process for the Preparation of Dolutegravir. 2018/0244693 A1, 2018.

(11) Maras, N.; Selic, L.; Cusak, A. Process for Preparing Dolutegravir and Cabotegravir and Analogues Thereof. 2017/0368040 A1, 2017.

(12) Schreiner, E.; Richter, F.; Nerdinger, S. Development of Synthetic Routes to Dolutegravir. In Top Heterocycl Chem; Springer International Publishing: Switzerland, 2016; Vol. 44, pp 187-208. doi.org/10.1007/7081_2016_200.

(13) Johns, B. A.; Kawasuji, T.; Weatherhead, J. G.; Taishi, T.; Temelkoff, D. P.; Yoshida, H.; Akiyama, T.; Taoda, Y.; Murai, H.; Kiyama, R.; et al. Carbamoyl Pyridone HIV-1 Integrase Inhibitors 3. A Diastereomeric Approach to Chiral Nonracemic Tricyclic Ring Systems and the Discovery of Dolutegravir (S/GSK1349572) and (S/GSK1265744). J. Med. Chem. 2013, 56 (14), 5901-5916. doi.org/10.1021/jm400645w.

(14) Yoshida, H.; Kawasuji, T.; Taoda, Y. Bicyclic Carbamoyl Pyridone Derivative Having HIV Integrase Inhibitory Activity. US 7,858,788 B2, 2016.

(15) Hughes, D. L. Review of Synthetic Routes and Final Forms of Integrase Inhibitors Dolutegravir, Cabotegravir, and Bictegravir. Org. Process Res. Dev. 2019, 6, acs.oprd.9b00031. doi.org/10.1021/acs.oprd.9b00031.

(16) Yasukata, T.; Masui, M.; Ikarashi, F.; Okamoto, K.; Kurita, T.; Nagai, M.; Sugata, Y.; Miyake, N.; Hara, S.; Adachi, Y.; et al. Practical Synthetic Method for the Preparation of Pyrone Diesters: An Efficient Synthetic Route for the Synthesis of Dolutegravir Sodium. Org. Process Res. Dev. 2019, 23 (4), 565-570. doi.org/10.1021/acs.oprd.8b00410.

(17) Lerner, C.; Kreis L.; Hilpert, H. Pyrimidone Derivatives and Their Use in the Treatment, Amelioration or Prevention of Viral Disease. WO2017158151, 2017.

(18) Kawasuji, T.; Fuji, M.; Yoshinaga, T.; Sato, A.; Fujiwara, T.; Kiyama, R. A Platform for Designing HIV Integrase Inhibitors. Part 2: A Two-Metal Binding Model as a Potential Mechanism of HIV Integrase Inhibitors. Bioorganic Med. Chem. 2006, 14 (24), 8420-8429. doi.org/10.1016/j.bmc.2006.08.043.

(19) Johns, B. A.; Svolto, A. C. Advances in Two-Metal Chelation Inhibitors of HIV Integrase. Expert Opin. Ther Pat. 2008, 18 (11), 1225-1237. doi.org/10.1517/13543776.18.11.1225.

We claim:

1. A compound of the formula: 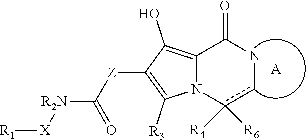
wherein,
ring A is an optionally substituted heterocycle;
Z is selected from the group consisting of: 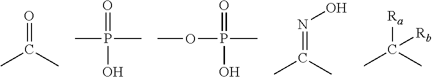
X is selected from the group consisting of:
a single bond;
a heteroatom or a heteroatomic group selected from the group consisting of O, S, SO, SO₂ and NH;
a C ₁-C₆ alkylene;
a C₁-C₆ heteroalkylene;
a C₂-C₆ alkenylene; and
C₂-C₆ heteroalkenylene;
Rₐ and R_{b} are independently substituted hydrogen, OH, C₁-C₁₀ alkyl or C₁-C₁₀ alkoxide;
R₁ is an optionally substituted aryl;
R₂ is a hydrogen or optionally substituted C₁-C ₁₀ alkyl;
R₃ is selected from the group consisting of hydrogen, a halogen, a hydroxy, an optionally substituted C₁-C₁₀ alkyl, an optionally substituted C₃-C₈ cycloalkyl, an optionally substituted C₂-C₈ alkenyl, an optionally substituted C₁-C₁₀ alkoxy, an optionally substituted C₂-C₈ alkenyloxy, an optionally substituted aryl, optionally substituted aryloxy, an optionally substituted heterocyclic group, an optionally substituted heterocycleoxy, an optionally substituted amino, an optionally substituted carbamoyl, and an optionally substituted carbamoylcarbonyl;
R₄ and R₆ are independently hydrogen, a hydroxyl, an optionally substituted C₁-C₁₀ alkyl, an optionally substituted C₁-Cₜₒ heteroalkyl, an optionally substituted C₂-C₈ alkenyl, an optionally substituted C₁-C₁₀ alkoxy, an optionally substituted C₂-C₈ alkenyloxy, an optionally substituted aryl, an optionally substituted aryloxy, an optionally substituted heterocycle, an optionally substituted heterocycleoxy, an optionally substituted amino, an optionally substituted carbamoyl, an optionally substituted carbamoylcarbonyl, or an optionally substituted phosphoric acid moiety, or
R₄ and R₆ are each part of an optionally substituted C₃-C₈ cycloalkyl, an optionally substituted heteroalkyl, an optionally substituted aryl, or an optionally substituted heteroaryl; [symbol]
represents a single or double bond, wherein if [symbol] is a double bond, then R₆ is not present;
or a pharmaceutically acceptable salt or solvate thereof.

2. The compound of claim 1 wherein at least one of R₄ and R₆ is a substituted aryl, aralkyl, C₁-C₁₀ alkyl, C₁-C ₁₀ heteroalkyl, hydroxyl, or amino.

3. The compound of claim 1, wherein at least one of R₄ and R₆ comprise a substituted or unsubstituted C₁-C₁₀ heteroalkyl and the substituted or unsubstituted C₁-C₁₀ heteroalkyl comprises one or more of O, S, SO, SO₂, or NR₅ wherein
R₅ is selected from the group consisting of hydrogen, an optionally substituted C₁-C₁₀ alkyl, an optionally substituted cycloalkyl, an optionally substituted C₂-C₈ alkenyl, an optionally substituted C₁-C₁₀ alkoxy, an optionally substituted aryl, an optionally substituted aryloxy, an optionally substituted heterocyclic group, an optionally substituted heterocycleoxy, an optionally substituted amino, an optionally substituted carbamoyl, an optionally substituted carbamoylcarbonyl, and an optionally substituted phosphoric acid moiety.

4. The compound of claims 1-3 comprising at least one optionally substituted aryl that is an optionally substituted aryl C₁-C₁₀ alkyl.

5. The compound of claims 1-3 comprising at least one optionally substituted heterocyclic ring that is a substituted heterocycle C₁-C₁₀ alkyl.

6. The compound of claims 1-3 comprising at least one optionally substituted amino or carbamoyl or carbamoylcarbonyl that is an amino or carbamoyl or carbamoylcarbonyl group which is unsubstituted or substituted by a group selected from mono- or di-C₁-C₁₀ alkyl, C₁-C₁₀ alkylcarbonyl, C₁-C₁₀ alkylsulfonyl, optionally substituted C₁-C₁₀ alkyl, carbamoylalkyl, mono- or di-C₁-C₁₀ alkylcarbamoyl, hydroxyl C₁-C₁₀ alkyl, heterocycle C₁-C₁₀ alkyl, C₁-C₁₀ alkoxycarbonyl C₁-C₁₀ alkyl, mono- or di-C₁-C₁₀ alkylamino C₁-C₁₀ alkyl, C₁-C₁₀ alkoxy C₁-C₁₀ alkyl, acyl, C₁-C₁₀ alkoxy C₁-C₁₀ alkylcarbonyl, C₁-C₁₀ alkylcarbamoyl alkylcarbonyl, C₁-C₁₀ alkoxycarbonylacetyl, optionally substituted arylcarbonyl, optionally substituted aralkyl, hydroxy, optionally substituted C₁-C₁₀ alkylsulfonyl, C₁-C₁₀ alkyl, or arylsulfonyl optionally substituted with halogen, C₃-C₈ cycloalkyl, aryl optionally substituted with C₁-C₁₀ alkyl, C₁-C₁₀ alkylaminosulfonyl, alkylaminocarbonyl, C₁-C₁₀ alkoxycarbonyl, C₃-C₈ cycloalkylcarbonyl, optionally substituted sulfamoyl, alkylcarbonylamino, heterocycle, and optionally substituted amino, and
wherein, as to the amino of an optionally substituted amino, an optionally substituted carbamoyl, or an optionally substituted carbamoylcarbonyl, two substituents on the amino together with the neighboring N atom may form an N-containing heterocycle which optionally contains S and/or O in the ring and is optionally substituted with oxo or hydroxyl.

7. The compound of claims 1-3 comprising at least one group which is optionally substituted, other than an optionally substituted amino, an optionally substituted carbamoyl, or an optionally substituted phosphoric acid,
wherein the at least one group is unsubstituted or substituted at any position by 1 to 4 substituents B, which are the same or different, B being selected from the group consisting of a hydroxy, a carboxy, a halogen, a halo C₁-C₁₀ alkyl, a halo C₁-C₁₀ alkoxy, a C₁-C₁₀ alkyl, a C₂-C₈ alkenyl, an ethynyl, a C₃-C₈ cycloalkyl, a cycloalkenyl, a C₁-C₁₀ alkoxy C₂-C₈ alkenyloxy, a C₁-C₁₀ alkoxycarbonyl, a nitro, a nitroso, an acylamino, an aralkylamino, a hydroxyamino, an azido, an aryl, an aralkyl, a cyano, an isocyano, an isocyanate, a thiocyanate, an isothiocyanate, a mercapt, an alkylthio, an alkylsulfonyl, an optionally substituted alkylsulfonylamino, am optionally substituted carbamoyl, a sulfamoyl, an acyl, a formyloxy, a haloformyl, an oxal, a thioformyl, a thiocarboxy, a dithiocarboxy, a thiocarbamoyl, a sulfino, a sulfo, a sulfoamino, a hydrazino, a ureido, an amizino, a quanidino, a phthalimide, an oxo, a phosphoric acid moiety, a C₁-C₁₀ alkyl which is substituted with a phosphoric acid moiety and which is either intervened or not intervened with one or more heteroatom groups.

8. The compound of claim 1 having a formula: 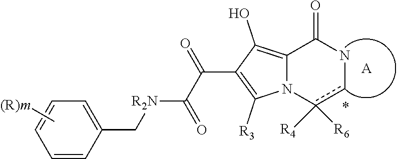
wherein,
* indicates an R- or S-configuration,
R is independently selected from halogen and and a substituent group S1;
wherein substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C₁-C₁₀ alkyl, C₁-C₁₀ alkoxy, carbamoyl optionally substituted with mono- or di-C₁-C₁₀ alkyl, optionally substituted C₁-C₁₀ alkyl sulfonyl amino, halogenated C₁-C₁₀ alkoxy and hydroxy C₁-C₁₀ alkyl; and
m is 0, 1, 2, or 3.

9. The compound of claim 8, wherein the optionally substituted C₁-C₁₀ heteroalkyl comprises at least one of CO, O, S, SO, SO₂ or NR^{a} where R^{a} is hydrogen, OH or C₁-C₁₀ alkyl.

10. A compound of claim 8, wherein R₂ is a hydrogen.

11. A compound according to any of the preceding claims, pharmaceutically acceptable salt, or solvate thereof, wherein ring A is any one of the following: 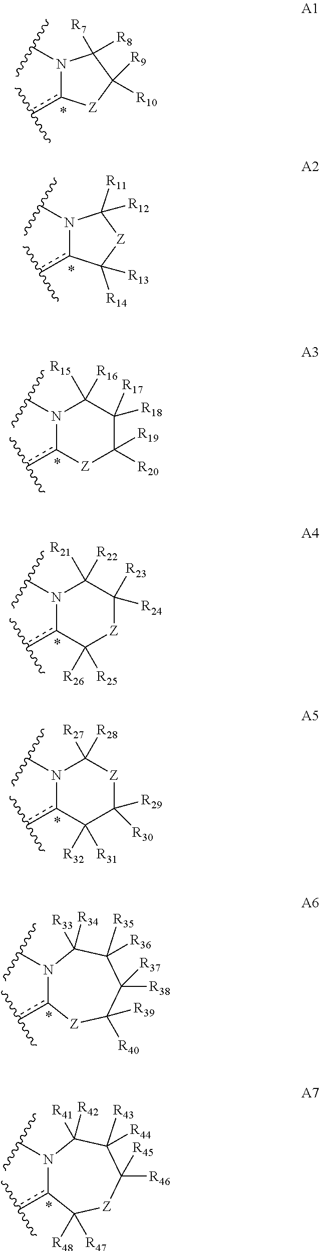 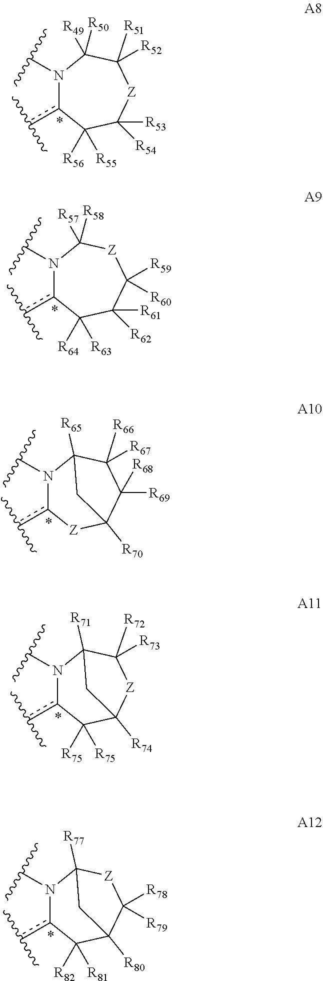
wherein
i) R₇ to R₈₇ are each independently selected from the group consisting of: hydrogen, an optionally substituted C₁-C₁₀ alkyl, an optionally substituted C₃-C₈ cycloalkyl, an optionally substituted C₂-C₈ alkenyl, an optionally substituted C₁-C₁₀ alkoxy, an optionally substituted C₂-C₈ alkenyloxy, an optionally substituted aryl, an optionally substituted aryloxy, a first optionally substituted heterocycle, an optionally substituted heterocycleoxy, hydroxy, and an optionally substituted amino; and/or
ii) one or more of R₇ to R₈₂ form part of an optionally substituted carbocycle or an optionally substituted heterocarbocycle; and/or
iii) Z is O or NR₈₃, wherein R₈₃ is hydrogen or C₁-C₁₀ alkyl.

12. The compound of claim 10, wherein the optionally substituted aryl is an optionally substituted aryl C₁-C₁₀ alkyl; the optionally substituted heterocycle is an optionally substituted heterocycle C₁-C₁₀ alkyl; and/or the optionally substituted C₃-C₈ cycloalkyl is an optionally substituted C₃-C₈ cycloalkyl C₁-C₁₀ alkyl.

13. The compound of claim 10, wherein any two of R₇ to R₈₂ which are attached to the same ring carbon atom, together with the carbon atom to which they are attached, form the optionally substituted carbocycle or the optionally substituted heterocarbocycle.

14. The compound of claim 10, wherein R₇ to R₈₂ are each independently hydrogen or an optionally substituted C₁-C₁₀ alkyl.

15. The compound of claim 10, further comprising one or more optionally substituted carbocycles or optionally substituted heterocarbocycles wherein at least one of the one or more optionally substituted carbocycles or optionally substituted heterocarbocycles comprises 3-7 ring atoms.

16. The compound of claim 10, wherein ring A is a ring represented by A1 and R₇ to R₁₀ are independently a hydrogen or an optionally substituted C₁-C₁₀ alkyl.

17. The compound of claim 10, wherein ring A is a ring represented by A2 and R₁₁ to R₁₄ are independently a hydrogen or an optionally substituted C₁-C₁₀ alkyl.

18. The compound of claim 10, wherein ring A is a ring represented by A3 and R₁₃ to R₂₀ are independently hydrogen or an optionally substituted C₁-C₁₀ alkyl.

19. The compound of claim 10, wherein ring A is a ring represented by A4 and R₂₁ to R²⁶ are independently hydrogen or an optionally substituted C₁-C₁₀ alkyl.

20. The compound of claim 10, wherein ring A is a ring represented by A5 and R₂₇ to R₃₂ are independently hydrogen or an optionally substituted C₁-C₁₀ alkyl.

21. The compound of claim 10, wherein ring A is a ring represented by A6 and R₃₃ to R₄₀ are independently hydrogen or an optionally substituted C₁-C₁₀ alkyl.

22. The compound of claim 10, wherein ring A is a ring represented by A7 and R₄₁ to R₄₈ are independently hydrogen or an optionally substituted C₁-C₁₀ alkyl.

23. The compound of claim 10, wherein ring A is a ring represented by A8 and R₄₉ to R₅₆ are independently hydrogen or an optionally substituted C₁-C₁₀ alkyl.

24. The compound of claim 10, wherein ring A is a ring represented by A9 and R₅₇ to R₆₄ are independently hydrogen or an optionally substituted C₁-C₁₀ alkyl.

24. The compound of claim 10, wherein ring A is a ring represented by A10 and R₆₅ to R₇₀ are independently hydrogen or an optionally substituted C₁-C₁₀ alkyl.

25. The compound of claim 10, wherein ring A is a ring represented by A11 and R₇₁ to R₇₆ are independently hydrogen or an optionally substituted C₁-C₁₀ alkyl.

26. The compound of claim 20, wherein ring A is a ring represented by A12 and R₇₇ to R₈₂ are independently hydrogen or an optionally substituted C₁-C₁₀ alkyl.

27. The compound of claim 1 has any of these formula II has any of these formulas:

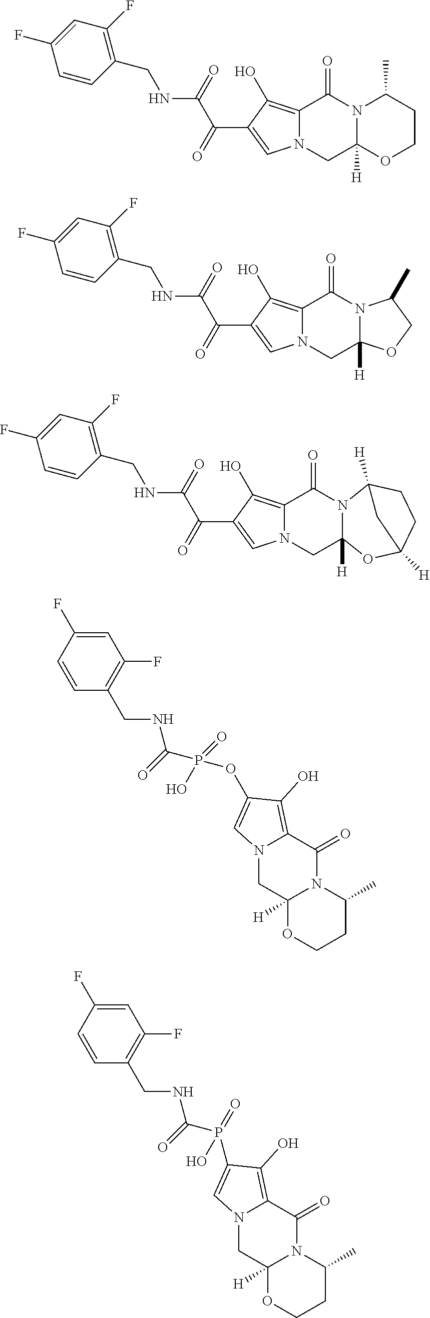 [SMILES: C[C@@H]1CCO[C@H]2Cn3cc(C(=O)C(=O)NCc4ccc(F)cc4F)c(O)c3C(=O)N12 ; C[C@H]1CO[C@@H]2Cn3cc(C(=O)C(=O)NCc4ccc(F)cc4F)c(O)c3C(=O)N12 ; O=C(NCc1ccc(F)cc1F)C(=O)c1cn2c(c1O)C(=O)N1[C@H]3CC[C@H](C3)O[C@@H]1C2 ; C[C@@H]1CCO[C@H]2Cn3cc(OP(=O)(O)C(=O)NCc4ccc(F)cc4F)c(O)c3C(=O)N12 ; C[C@@H]1CCO[C@H]2Cn3cc(P(=O)(O)C(=O)NCc4ccc(F)cc4F)c(O)c3C(=O)N12]

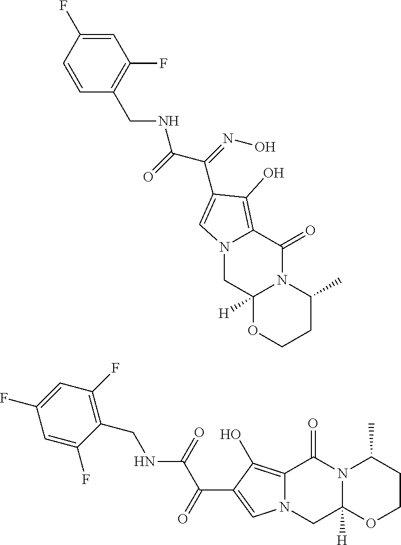 [SMILES: C[C@@H]1CCO[C@H]2Cn3cc(/C(=N\O)C(=O)NCc4ccc(F)cc4F)c(O)c3C(=O)N12 ; C[C@@H]1CCO[C@H]2Cn3cc(C(=O)C(=O)NCc4c(F)cc(F)cc4F)c(O)c3C(=O)N12]

28. A pharmaceutical composition comprising a compound according to any one of claims 1 to 27, or a pharmaceutically acceptable salt, or solvate thereof.

29. A process for preparing a compound of formula II 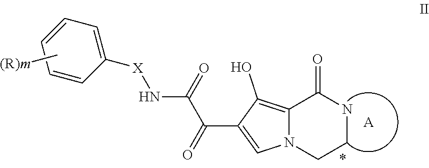
comprising:
providing a compound of formula 15 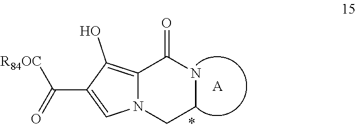
providing a compound of formula 16 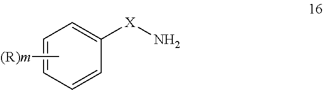
and reacting the compound of formula 15 with the compound of formula 16
wherein,
A is an optionally substituted 3-7 carbon heterocycle,
* indicates an R- or S-configuration,
X is selected from the group consisting of
a single bond;
a heteroatom or a heteroatomic group selected from the group consisting of O, S, SO, SO₂ and NH;
C₁-C₆ alkylene;
C₁-C₆ heteroalkylene;
C₂-C₆ alkenylene; and
C₂-C₆ heteroalkenylene;
R is independently selected from halogen and Substituent group S1;
wherein substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C₁-C₁₀ alkyl, C₁-C₁₀ alkoxy, a carbamoyl optionally substituted with mono- or di-C₁-C₁₀ alkyl, an optionally substituted C₁-C₁₀ alkyl sulfonyl amino, halogenated C₁-C₁₀ alkoxy and hydroxy C₁-C₁₀ alkyl;
m is 0, 1, 2, or 3, and
R₈₄ is hydrogen or C ₁-C₁₀ alkyl.

30. A process according to claim 29, wherein the step of providing the compound of formula 15 is performed by
reacting compound 17 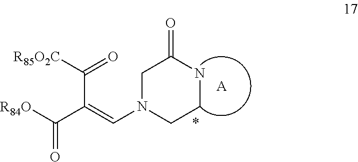
with a base and a Mg salt
wherein,
A is an optionally substituted 3-7 carbon heterocycle,
* indicates an R- or S-configuration,
R₈₄ and R₈₅ are hydrogen or C₁-C₁₀ alkyl,
the base is Et₃N, DMAP, DBU, pyridine, LiHMDS, CaO, DIPEA, K₃PO₄, LDA, Ca(OMe)₂, LiOMe, NaOMe and
the magnesium salt is Mg(OMe)₂ or Mg(OtBu)₂, MgBr₂.OEt₂. MgO, MgClO₄, MgX₂ (where X ═F, Cl, Br, I).

31. A process according to claim 30, wherein the step of providing compound of formula 17 is performed by
reacting a compound of formula 18, 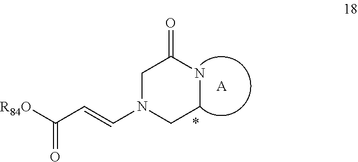
with a compound of formula 19, 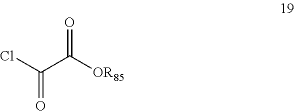
wherein,
A is an optionally substituted 3-7 carbon membered heterocycle,
* indicates an R- or S-configuration, and
R₈₄ and R₈₅ are hydrogen or C₁-C₁₀ alkyl.

32. A process according to claim 31, wherein the step of providing compound of formula 18 is performed by
reacting a compound of formula 10, 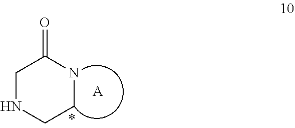
with compound of formula 8, 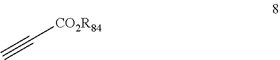
wherein,
A is an optionally substituted 3-7 carbon heterocycle,
* indicates an R- or S-configuration, and
R₈₄ is hydrogen or C₁-C₁₀ alkyl.

33. A process of preparing a compound of formula II-a, 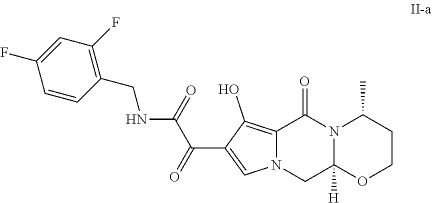 [SMILES: C[C@@H]1CCO[C@H]2Cn3cc(C(=O)C(=O)NCc4ccc(F)cc4F)c(O)c3C(=O)N12]
comprising,
i) reacting a compound of formula 10-a 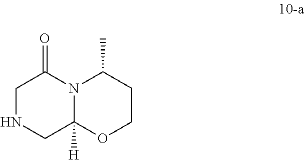
with 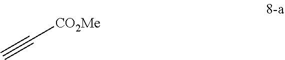
to form intermediate 18-a 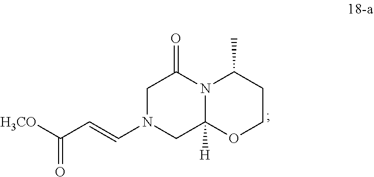 [SMILES: COC(=O)/C=C/N1CC(=O)N2[C@H](C)CCO[C@H]2C1]
ii) reacting intermediate 18-a with 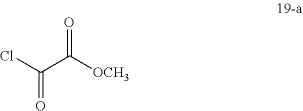
to form intermediate 17-a 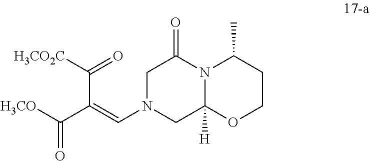 [SMILES: COC(=O)C(=O)/C(=C\N1CC(=O)N2[C@H](C)CCO[C@H]2C1)C(=O)OC]
iii) reacting intermediate 17-a with a combination of a magnesium salt and a base to form intermediate 15-a 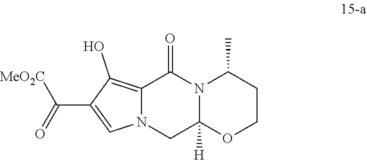
wherein,
the magnesium salt is Mg(OMe)₂ or Mg(OtBu)₂, MgBr₂.OEt_{2.} MgO, MgClO₄, MgX₂ (where X ═F, Cl, Br, I)
the base is Et₃N, DMAP, DBU, pyridine, LiHMDS, CaO, DIPEA, K₃PO₄, LDA, Ca(OMe)₂, LiOMe, NaOMe;
and
iv) reacting intermediate 15-a with 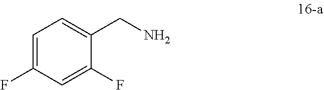
to form compound II-a.

34. A process for preparing a compound of formula 17 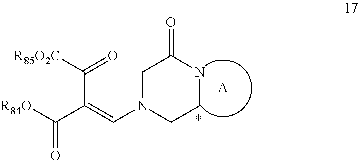
comprising
i) reacting a compound of formula 10 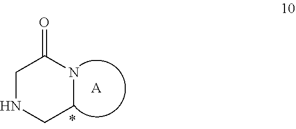
with a compound of formula 8, 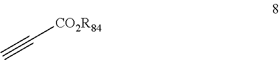
to form an intermediate of formula 18, 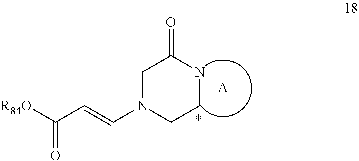
ii) reacting an intermediate of formula 18
reacting a compound of formula 18, 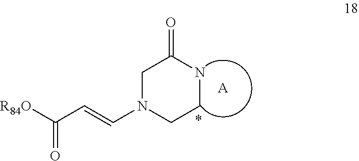
with a compound of formula 19, 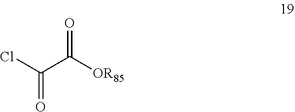
to form a compound of formula 17
wherein,
A is an optionally substituted 3-7 carbon heterocycle,
* indicates an R- or S-configuration,
R₈₄ and R₈₅ are hydrogen or C₁-C₁₀ alkyl, and
the base is Et₃N, DMAP, DBU, pyridine, LiHMDS, CaO, DIPEA, K₃PO₄, LDA, Ca(OMe)₂, LiOMe, NaOMe.

35. A process for preparing a compound of formula 20 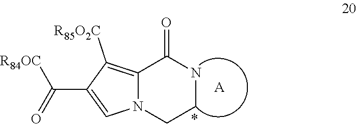
comprising of
reacting compound 17 with a base 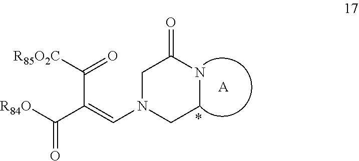
wherein,
A is an optionally substituted 3-7 carbon heterocycle,
* indicates an R- or S-configuration,
R₈₄ and R₈₅ are hydrogen or C₁-C₁₀ alkyl, and
the base is Et₃N, DMAP, DBU, pyridine, LiHMDS, CaO, DIPEA, K₃PO₄, LDA, Ca(OMe)₂, LiOMe, NaOMe.

36. A process of preparing dolutegravir of formula I 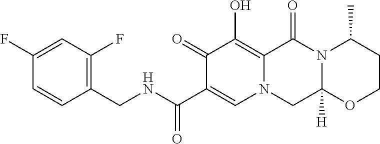 [SMILES: C[C@@H]1CCO[C@H]2Cn3cc(C(=O)NCc4ccc(F)cc4F)c(=O)c(O)c3C(=O)N12]
comprising,
i) reacting 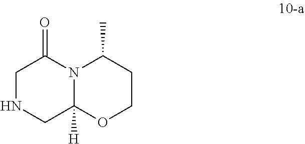
with 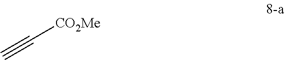
to form Intermediate 18-a 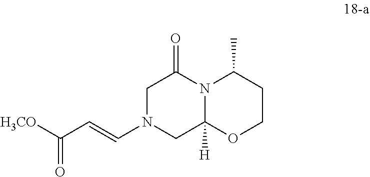 [SMILES: COC(=O)/C=C/N1CC(=O)N2[C@H](C)CCO[C@H]2C1]
ii) reacting 18-a with 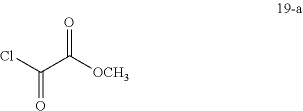
to form Intermediate 17-a 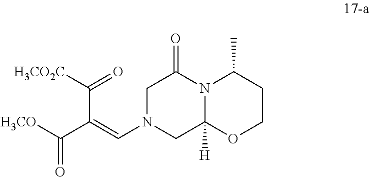 [SMILES: COC(=O)C(=O)/C(=C\N1CC(=O)N2[C@H](C)CCO[C@H]2C1)C(=O)OC]
iii) reacting 17-a with a combination of a magnesium salt and a base to form Intermediate 7 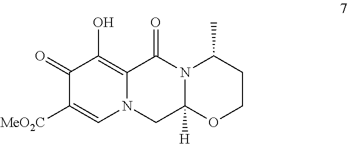
wherein,
the magnesium salt is Mg(OMe)₂ or Mg(OtBu)₂, MgBr₂.OEt₂, MgO, MgClO₄, MgX₂ (where X =F, Cl, Br, I).
the base is Et₃N, DMAP, DBU, pyridine, LiHMDS, CaO, DIPEA, K₃PO₄, LDA, Ca(OMe)₂, LiOMe, NaOMe.
and
iv) reacting Intermediate 7 with 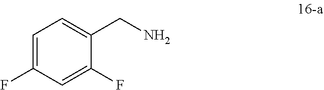
to form dolutegravir.

37. A compound of formula 17: 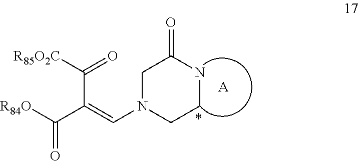
wherein,
A is an optionally substituted 3-7 carbon heterocycle,
* indicates an R- or S-configuration,
R₈₄ and R₈₅ are hydrogen or C₁-C₁₀ alkyl.

38. A compound of formula 17-a: 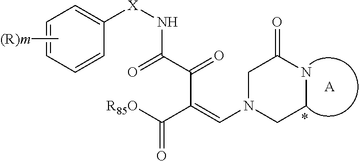
where,
A is an optionally substituted heterocycle or heterocarbocycle;
* indicates an R- or S-configuration,
X is selected from the group consisting of a single bond; a heteroatom or a heteroatomic group selected from the group consisting of O, S, SO, SO₂ and NH; C₁-C₆ alkylene; C₁-C₆ heteroalkylene; C₂-C₆ alkenylene; and C₂-C₆ heteroalkenylene,
R₈₅ are hydrogen or C₁-C₁₀ alkyl,
R is independently selected from halogen and and a substituent group S1;
wherein substituent group S1 is selected from: an optionally substituted phosphoric acid moiety, an aryl substituted with an optionally substituted phosphoric acid moiety, an aralkyl substituted with an optionally substituted phosphoric acid moiety, a hydroxy substituted with an optionally substituted phosphoric acid moiety, an amino substituted with an optionally substituted phosphoric acid moiety, halogenated C₁-C₁₀ alkyl, C₁-C₁₀ alkoxy, carbamoyl optionally substituted with mono- or di-C₁-C₁₀ alkyl, optionally substituted C₁-C₁₀ alkyl sulfonyl amino, halogenated C₁-C₁₀ alkoxy and hydroxy C₁-C₁₀ alkyl; and
m is 0, 1, 2, or 3.

39. A compound, or a pharmaceutically acceptable salt, or solvate as defined in any one of claims 1 to 27 and 38 for use as an antiviral agent.

40. A compound, or a pharmaceutically acceptable salt, or solvate as defined in any one of claims 1 to 27 and 38 for use as an anti-HIV agent.

41. A compound, or a pharmaceutically acceptable salt, or solvate as defined in any one of claim 38 for use as an anti-HIV agent.

42. A compound of formula 18: 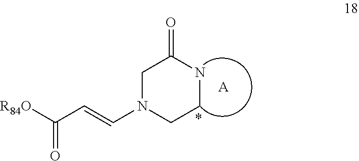
wherein,
A is an optionally substituted 3-7 carbon heterocycle,
* indicates an R- or S-configuration,
R₈₄ is hydrogen or C₁-C₁₀ alkyl.

43. A compound of formula 20: 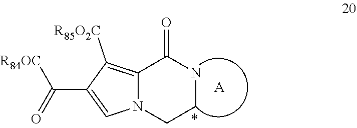
wherein,
A is an optionally substituted 3-7 carbon heterocycle,
* indicates an R- or S-configuration,
R₈₄ and R₈₅ are hydrogen or C₁-C₁₀ alkyl.
